(19) United States
(12) Patent Application Publication
(10) Pub. No.: US 2007/0148194 A1
(43) Pub. Date: Jun. 28, 2007

(54) Novel nanoemulsion formulations

(76) Inventors: Mansoor M. Amiji, Attleboro, MA (US); Sandip B. Tiwari, North Wales, PA (US)

Correspondence Address: WEINGARTEN, SCHURGIN, GAGNEBIN & LEBOVICI LLP, TEN POST OFFICE SQUARE, BOSTON, MA, 02109, US

(21) Appl. No.: 11/606,641

(22) Filed: Nov. 29, 2006

Related U.S. Application Data
(60) Provisional application No. 60/740,602, filed on Nov. 29, 2005.

(51) Int. Cl. A61K 9/00

(52) U.S. Cl. 424/400

(57) ABSTRACT

An oil-in-water nanoemulsion delivery system that includes at least one oil having a concentration of greater than or equal to 2% (w/w) of at least one polyunsaturated fatty acid, preferably of the omega-3 or omega-6 family, is disclosed. The delivery system further includes at least one emulsifier and also an aqueous phase. Preferably, one or more hydrophobic therapeutic, monitoring and/or diagnostic agents are dispersed in the oil phase. The nanoemulsions may optionally contain other conventional pharmaceutical aids such as stabilizers, preservatives, buffering agents, antioxidants, polymers, proteins and charge inducing agents. The invention also relates to a process for preparing the nanoemulsions and to their use in the oral, parenteral, opthalmic, nasal, rectal or topical delivery of hydrophobic therapeutic, monitoring or diagnostic agents.

### CROSS REFERENCE TO RELATED APPLICATIONS

This application claims the priority of U.S. Provisional Application No. 60/740,602 filed Nov. 29, 2006, entitled, NOVEL NANOEMULSION FORMULATIONS, the whole of which is hereby incorporated by reference herein.

### STATEMENT REGARDING FEDERALLY SPONSORED RESEARCH OR DEVELOPMENT

Part of the work leading to this invention was carried out with United States Government support provided by the National Cancer Institute of the National Institutes of Health under grants R01-CA-095522 and R01-CA-119617. Therefore, the U.S. Government has certain rights in this invention.

### BACKGROUND OF THE INVENTION

Conventional self-emulsifying drug delivery formulations are widely used in enhancing the oral absorption of poorly soluble drug (Charman, 2000; Pouton, 2000; Bagwe et al., 2001; Wasan et al., 2001; Gursoy et al., 2003). Hydrophobic drugs can be dissolved in such systems, allowing them to be encapsulated as unit dosage forms for oral administration. Exemplary systems of the prior art are isotropic mixtures of oil and surfactants, which can disperse in the gastrointestinal (GI) tract to form microemulsion (droplet size less than 30 nm), fine opaque emulsions (droplet size 50 to 200 nm), or coarse emulsions with droplet size of larger than 500 nm upon dilution with GI fluid (Gusoy et al., 2003). A drug administered in this manner remains in solution in the GI tract, avoiding the dissolution step, which frequently limits the rate of absorption of hydrophobic drugs from the crystalline state (Pouton, 1997). However, the high surfactant level typically present in such formulations can lead to GI side-effects as well as to a reduction in the free drug concentration and, thus, a reduced rate of intestinal absorption (Poelma et al., 1991).

Other approaches for administering hydrophobic drugs include the use of co-solvents, incorporation of complexing or solubilising agent, chemical modification of the drug, use of micellar systems such as niosomes or liposomes or their formulation in an oily vehicle for either oral, parenteral, nasal, rectal, ophthalmic, rectal or ophthalmic delivery. However, many of these formulations also employ surfactants/co-solvents having associated toxic side effects. Liposomes have been examined by various researchers as delivery vehicle for both water soluble and water insoluble drugs. With most of such systems, though, stability, sterility and mass commercial production issues are common. Most importantly, the existing delivery formulations for hydrophobic drugs have a limited ability to overcome the different transport barriers in biological systems, the blood/brain barrier being one of the most important.

Yet, improving the delivery of hydrophobic compounds as pharmaceutic agents continues to be an area of great interest from a commercial point of view. Almost one third of the drugs in the United States Pharmacopoeia are either insoluble or poorly soluble. Also increasing numbers of potential new chemical entities are being dropped in the early phases of development because of poor solubility or insolubility. Any improvement to the delivery method of these hydrophobic agents (either in terms of efficiency or targetability) would result in an improvement in terms of healthcare management.

### BRIEF SUMMARY OF THE INVENTION

Nanoemulsions or mini-emulsions are dispersions of oil in a water phase with the oil droplets confined to the nanometer size range. As they are biphasic systems, they differ from microemulsion (Salager, 2000). Nanoemulsion formulations are capable of reducing surfactant side-effects and yet still achieving enhanced oral bioavailability of lipophilic drugs. The system amd method of the invention are directed to an improved nanoemulsion formulation for delivery of hydrophobic compounds that incorporates preferred fatty acids in the oil phase.

Thus, the present invention is directed to a novel oil-in-water nanoemulsion delivery system that includes at least one oil having a concentration of greater than or equal to 2% (w/w) of at least one polyunsaturated fatty acid, preferably of the omega-3 or omega-6 family. The system further includes at least one emulsifier and also an aqueous phase. Preferably, one or more hydrophobic therapeutic, monitoring and/or diagnostic agents is dispersed in the oil phase. The nanoemulsions may optionally contain other conventional pharmaceutical aids such as stabilizers, preservatives, buffering agents, antioxidants, polymers, proteins and charge inducing agents. The invention also relates to a process for preparing the nanoemulsions and to their use in the oral, parenteral, opthalmic, nasal, rectal or topical delivery of hydrophobic therapeutic, monitoring or diagnostic agents.

Thus, the invention expands the use of nanoemulsions as transport systems for pharmaceutic agents to encompass the delivery of such agents through biological membranes or barrier systems. The nanoemulsions of the present invention can be tailored depending on the agent to be incorporated and the route of administration.

### BRIEF DESCRIPTION OF THE SEVERAL VIEWS OF THE DRAWINGS

Other features and advantages of the invention will be apparent from the following description of the preferred embodiments thereof and from the claims, taken in conjunction with the accompanying drawings, in which:

FIG. 1 shows the whole blood concentration-time profile of tritiated [³H] paclitaxel in female C57BL/6 mice following oral administration of the control and nanoemulsion formulations. Legends are—control solution=commercial paclitaxel solution for injection diluted with saline; NE-standard=paclitaxel-loaded nanoemulsions with egg phosphatidylcholine as emulsifier; NE-stearylamine=paclitaxel-loaded nanoemulsions with the cation stearylamine as a co-emulsifier in addition to egg phosphatidylcholine; NE-deoxycholic acid=paclitaxel-loaded nanoemulsions with the anion deoxycholic acid as a co-emulsifier in addition to egg phosphatidylcholine;

FIG. 2 shows the in vivo biodistribution profile of paclitaxel nanoemulsions according to the invention at the 1 hour, 6 hour, 12 hour and 24 hour time points at the indicated tissue sites. The legends are according to those given for FIG. 1;

FIGS. 3A and 3B are plasma (3A) and brain (3B) saquinavir concentration versus time profiles following oral administration of the drug saquinavir in aqueous suspension or nanoemulsion formulations to Balb/c mice;

FIGS. 4A and 4B are plasma (4A) and brain (4B) saquinavir concentration versus time profiles following intravenous administration of the drug saquinavir in aqueous suspension or nanoemulsion formulations to Balb/c mice;

FIGS. 5A and 5B are bar graphs showing the area-under-the curve (AUC_{0→∞}) data from saquinavir concentration versus time profiles in various harvested tissues to show the biodistribution profile following oral (5A) and intravenous (5B) administration in Balb/c mice. RIG denotes the rest of gastrointestinal tract, except stomach;

FIG. 6 is a schematic representation of a multifunctional nanoemulsion and also of the anatomy of one nanoparticle (e.g., oil globule or nanodroplet) in the nanoemulsion complexed with DTPA-PE-Gd⁺³;

FIG. 7 is a bar graph showing cell viability following incubation with paclitaxel based upon an MTS assay. NE=standard nanoemulsion without paclitaxel. SA-NE=standard nanoemulsion with stearylamine. NE+PTX=nanoemulsion containing paclitaxel. SA-NE-PTX=nanoemulsion with stearylamine and paclitaxel. PTX soln=aqueous solution of paclitaxel; and

FIGS. 8A, 8B, and 8C are graphs showing percent drug release with time at increasing temperature for nanoemulsions according to the invention having different emulsifiers.

### DETAILED DESCRIPTION OF THE INVENTION

The nanoemulsions in the delivery system of the present invention have unique features because of the novel excipients used in their manufacture. This delivery system can be tailored to overcome the biological barrier of a particular organ without toxic effects to the organs or tissues.

An essential component of the nanoemulsion system according to the invention is an oil phase comprising individual oil droplets, which represent the internal hydrophobic or oil core. The oil may be a single entity or mixture. Thus, the term “oil phase” as applied to the internal hydrophobic core herein may refer either to a single pure oil or to a mixture of different oils present in the core. The average size of oil droplets in the oil phase of the nanoemulsions described herein from may range from 5-500 nm.

Oil compositions suitable for use as the core component of nanoemulsions according to the invention may be characterized as being those containing high concentrations of essential polyunsaturated fatty acids (PUFA), preferably a concentration of greater than or equal to 10% (w/w) of at least one polyunsaturated fatty acid of the omega-3 or omega-6 family. Resources known to those of skill in the art may be used to identify such oils. Exemplary websites include the following:

www.lipidlibrary.co.uk/Lipids/fa_poly/index.htm
www.umm.edu/altmed/ConsSupplements/Omega3FattyAcidscs.html
curezone.com/foods/fatspercent.asp

In a preferred embodiment, suitable oils are those containing high concentrations of linoleic acid—for example, plant seed oils of evening primrose, black currant, pine nut or borage, and fungal oils such as spirulina and the like—either alone or in combination. The oils can be from any source rich in polyunsaturated fatty acids, preferably plant or animal sources. Chemically or enzymatically derivatized, or even completely synthetic, polyunsaturated fatty acids are included within the scope of suitable components for the oil phase of the delivery system of the invention. The concentration of the polyunsaturated fatty acid in the oil phase can range from 2% to 100% (w/w), more preferably from 5% to 100% (w/w) and most preferably can be greater than 10%, e.g., 20%-80% (w/w). The concentration of the oil phase, in the nanoemulsion preferably can vary from 5% w/w to 40% w/w, more preferably from 10% to 30% w/w.

The at least one (as used throughout herein above and below, the expression “at least one” means one or more and thus includes individual components as well as mixtures/combinations) emulsifier is present at the interphase between the oil phase and the aqueous phase. The emulsifier is preferably an amphiphilic molecule chosen from, for example, nonionic and ionic amphiphilic molecules. The expression “amphiphilic molecule” means any molecule of bipolar structure comprising at least one hydrophobic portion and at least one hydrophilic portion and having the property of reducing the surface tension of water (g<55 mN/m) and of reducing the interface tension between water and an oily phase. The synonyms of amphiphilic molecule are, for example, surfactant, surface-active agent and emulsifier.

In the preferred embodiment of present invention, the emulsifier can consist of neutral, positively charged, or negatively charged natural or synthetic phospholipids molecules such as natural phospholipids including soybean lecithin, egg lecithin, phosphatidylglycerol, phosphatidylinositol, phosphatidylethanolamine, phosphatidic acid, sphingomyelin, diphosphatidylglycerol, phosphatidylserine, phosphatidylcholine and cardiolipin; synthetic phospholipids including dimyristoylphosphatidylcholine, dimyristoylphosphatidylglycerol, distearoylphosphatidylglycerol and dipalmitoylphosphatidylcholine; and hydrogenated or partially hydrogenated lecithins and phospholipids. In preferred embodiments, phospholipids from natural source are used. Amphiphilic lipids can be used alone or in combination. The concentration of amphiphilic lipid in the nanoemulsions can vary from 0.5% to 15% (w/v), more preferably from 1% to 10% (w/v).

In addition to the emulsifier, charge-inducing agents, including but not limited to sterylamine, deoxycholic acid, and other bile-acids, are also included in the nanoemulsion as co-emulsifiers for specific applications.

An aqueous phase of the nanoemulsions can be either purified or ultrapure water, saline, buffered saline, buffered aqueous phase, glycerine, low molecular weight polyethylene glycol either alone or in combinations thereof. The concentration of aqueous phase in nanoemulsions can vary from 30% to 90% (w/w).

The nanoemulsions of the present invention optionally may contain other pharmaceutical aids, such as one or more stabilizers, antioxidants, preservatives, buffering agents, charge inducing agents, polymers and proteins. A preferred antioxidant is alpha-tocopherol or its derivatives, which are members of the Vitamin E family. Other antioxidants include butylated hydroxytoluene (BHT). Stabilizers can be pH modifying agents, anti-creaming or anti-foaming agents or agents helping to impart stability to the nanoemulsions. Representative examples of stabilizers include sodium oleate, glycerine, xylitol, sorbitol, ascorbic acid, sodium edetate. Representative charge inducing agents include sodium deoxycholate, deoxycholic acid, stearylamine, oleylamine, chitosan, cetyltriethylammonium bromide. Representative buffering agents include, sodium phosphate, citric acid, formic acid and the like.

One or more hydrophobic therapeutic, monitoring and/or diagnostic agents can be incorporated into the nanoemulsion delivery system according to the invention. The system of the invention is appropriate for delivery of multiple therapeutic agents, e.g., for combination therapy; for delivering a combination of a therapeutic and a monitoring agent; for delivering a combination of a therapeutic agent and an agent to enhance delivery or potency; or for delivering any combination of therapeutic, monitoring and/or diagnostic agents contemplated. Representative examples of therapeutic agents include but are not limited to non-steroid anti-inflammatory compounds, anti-neoplastic compounds, antibiotics, anti-convulsants, anti-epileptics, anti-fungals, glycosaminoglycans, hypnotics, vitamins, beta-adrenergic antagonists, anti-anxiety agents, tranquilizers, antidepressants, corticorsteroids, anabolic steroids, estrogens, and progesterones, proteins, peptides and other hormonal drugs, and drugs for the treatment of Alzheimer's disease and other disorders of the CNS. Exemplary monitoring or diagnostic agents include compounds used for magnetic resonance imaging contrast enhancement (e.g., gadolinium), computed tomography contrast enhancement (e.g., iodinated contrast agents), x-ray contrast enhancement, ultrasound contrast enhancement (e.g., via air entrapment), and optical imaging (e.g., using fluorescent probes); and radioactive isotopes for gamma and positron emission tomography imaging.

In general, the nanoemulsions of the present invention can be formulated by, first, preparing an aqueous phase containing an amphiphilic molecule and homogenizing this solution with a lab homogenizer or mixer for 10 min.; second, preparing an oil phase containing an oil, as above, and one or more hydrophobic therapeutic, monitoring and/or diagnostic agents, as desired, and mixing the same with suitable mixing device; and, third, heating the solutions of the first two steps at about 70° C. for about 10 min. or less, mixing these solutions together and then homogenized them with a probe sonicator (Sonics and Materials, USA) to obtain the desired nanoemulsion. Alternatively, a high pressure homogenizer (such as one made by Gauline or Avestine, or the like) or a microfludiser can be used for homogenization. The number of passes through a high pressure homogenizer/microfludiser can vary depending on the desired particle size for the nanoemulsions.

Alternatively, the oil phase can be mixed with a suitable organic solvent and the organic solvent evaporated to obtain an oil phase film. This film can then be hydrated with the aqueous solution described above and the mixture homogenized and sized (by a sonicator, a high pressure homogenizer or a microfludiser) to obtain the desired nanoemulsion. The pharmaceutical agent can be dissolved with the oil phase or added after the formation of the oil film along with the aqueous solution. To control the size distribution of the nanoemulsion oil droplets, the nanoemulsion can be filtered using, e.g., 0.45 micron membrane filters.

Nanoemulsions can be lyophilized, e.g., for preservation purposes, in the presence of suitable cryoprotectants. Examples of suitable cryoprotectants include, but are not limited to, glucose, manitol, glycine, high molecular weight polyethylene glycol and other cryoprotectants commonly used in lyophilization of pharmaceuticals. The concentration of cyoprotectants can vary from, e.g., 1% to 80% (w/v).

The nanoemulsion formulations of the present invention can be administered, e.g., as liquid emulsions or freeze dried along with a suitable cryoprotectant and filled in sachets or capsules or compressed into a tablet or caplet dosage form. Given the versatility of the nanoemulsion delivery system according to the inventionand depending on the types of excipients used, there are many potential applications of this system.

Therapeutic, monitoring and/or diagnostic compositions delivered via the nanoemulsion delivery system according to the invention may be administered orally, topically, or parenterally (e.g., intranasally, subcutaneously, intramuscularly, intravenously, or intra-arterially) by routine methods in pharmaceutically acceptable inert carrier substances. For example, the compositions of the invention may be administered in a sustained release formulation using a biodegradable biocompatible polymer, or by on-site delivery using micelles, gels or liposomes. The active agent delivered by the nanoemulsion system of the invention can be administered in a wide dosage range, e.g., 0.25 μg/kg/day to 5 mg/kg/day, and preferably 1 μg/kg/day to 500 μg/kg/day. Optimal dosage and modes of administration depend on the active agent to be delivered and can readily be determined by conventional protocols.

The following examples are presented to illustrate the advantages of the present invention and to assist one of ordinary skill in making and using the same. These examples are not intended in any way otherwise to limit the scope of the disclosure.

### EXAMPLE I

### Improved Oral Delivery of Paclitaxel

Paclitaxel is an important antitumor agent that is widely used in the treatment of advanced breast and ovarian cancer. The molecular weight of paclitaxel is 853 Da, and it has a very low aqueous solubility (<1 mg/mL (Lee et al., 2003; Mathew et al., 1992). Moreover, the compound does not contain any functional groups that can be ionized or that allow for salt formation to increase its aqueous solubility. The solubility of paclitaxel in other commonly used pharmaceutical vehicles and/or solvents (e.g., poly(ethylene glycol), propylene glycol, ethanol, etc.) is also limited. The development of a paclitaxel formulation that can be administered successfully has, therefore, been a challenge, and many approaches have been tested or are under investigation (Straubinger et al., 1993; Singla et al., 2002). The currently marketed intravenous formulation of paclitaxel (ONXOL™, Ivax Pharmaceuticals, Miami, Fla.) contains 6 mg/mL of paclitaxel, 527 mg/mL of Cremophor EL (polyoxyethylenated castor oil), and 49.7% (v/v) of dehydrated ethanol.

Although oral administration is the commonly preferred route for administration of pharmaceuticals, the oral bioavailability of paclitaxel is extremely low in animals and humans (Malingre et al., 2001; Bradelmeijer et al, 2000). As a result, there are no marketed oral paclitaxel products. Recently, preclinical studies in mice have shown that the low oral bioavailability of paclitaxel is due to the adverse effects of the multidrug efflux pump, P-glycoprotein (P-gp) that is abundantly present on the enterocyte membranes in the GI tract. Improved oral uptake of paclitaxel has been made possible in mice and humans by co-administration of oral cyclosporin A, an inhibitor of P-gp and cytochrome P-4503A4-mediated drug metabolism. In humans, co-administration of cyclosporin A resulted in a seven-fold increase in systemic exposure of paclitaxel, and plasma concentrations increased from negligible to therapeutic levels (Malingre et al., 2001; Bardelmeijer et al., 2001; Malingre et al., 2001). However, this approach will not be feasible in the clinic as the co-administration of P-gp-inhibiting immunosuppressants (such as cyclosporin A) with paclitaxel to cancer patients who are already immunodeficient as a result of chemotherapy will have catastrophic consequences.

In an effort to examine the applicability of the nanoemulsion formulations of the invention for improved oral delivery of hydrophobic drugs, paclitaxel was selected as a model hydrophobic P-gp substrate. The in vivo biodistribution studies of the paclitaxel nanoemulsion formulations, with or without charge inducing agents (deoxycholic acid or stearylamine), were conducted in mice and compared with the performance of the commercial paclitaxel formulation administered orally.

Materials

Paclitaxel solution for injection—ONXOL™ (6 mg/mL solution in 527 mg of polyoxyl 35 castor oil NF, 2 mg anhydrous citric acid, and 49.7% (v/v) dehydrated alcohol, USP)—was obtained from Ivax Pharmaceuticals, Inc. (Miami, Fla.). Tritiated [³H]-paclitaxel with an activity of 250 μCi in 250 μl ethyl alcohol was purchased from Moravek Biochemicals (Brea, Calif.). Pine nut oil was purchased from Siberian Tiger Naturals Inc. (Cabot, Vt.). Egg phosphatidylcholine (Lipoid® E80) was provided as a gift sample by Lipoid GMBH (Ludwigshfaen, Germany). The Lipoid® E80, according to manufacturer specifications, comprised about 80% phosphatidylcholine, 8% phosphatidylethanolamine, 3.6% non-polar lipids, and about 2% sphingomylein. Deoxycholic acid and stearylamine were purchased from Sigma Chemicals (St. Louis, Mo.). Deionized distilled water (Barnsted/Thermolyne, Dubuque, Iowa) was used exclusively for the preparation of all aqueous solutions.

Methods

Preparation of Paclitaxel-Loaded Nanoemulsions

A 20% oil-in-water nanoemulsion containing 420 μg/mL of paclitaxel was prepared by first dispersing an appropriate quantity of paclitaxel solution for injection (6 mg/mL; 1050 μl) in pine nut oil (3 mL) with stirring and gentle heating to 60-70° C. An appropriate volume of 36 aqueous solution of Lipoid® E80 (12 mL) was warmed to 70° C., combined with the oil solution of paclitaxel, and sonicated with a probe type sonicator (Sonics and Materials Inc., Vibra Cell VC 505, Newtown, Conn.) for 10 minutes at 21% amplitude and 50% duty cycle. The resulting dispersion was a uniform and milk-white color. The nanoemulsions were filtered using 0.45 μm membrane filter. Nanoemulsion formulations were also formulated using charge inducing co-surfactants such as deoxycholic acid (anionic) and stearylamine (cationic). In those formulations, the ratio of Lipoid® E80 to co-surfactants was maintained at 2:1 (w/w).

Characterization of the Resultant Nanoemulsions

Freshly prepared nanoemulsion formulations were analyzed for particle size and size distribution by the light scattering method using 90Plus® particle size analyzer from Brookhaven Instruments (Holtsville, N.Y.). The nanoemulsions were diluted suitably with deionized distilled water so as to obtain the average count rate of 50-500 kcps. Particle size analysis was carried out at a scattering angle of 90° and a temperature of 25° C.

Zeta potential (ξ) measurements of the nanoemulsion formulations were performed using the ZetaPALS® instrument (Brookhaven Corporation, Holtsville, N.Y.). The nanoemulsions were diluted with deionized distilled water and zeta potential values were measured at the default parameters of the dielectric constant, refractive index, and viscosity of water, and were calculated based on the electrophoretic mobility. The pH of the final diluted samples ranged from 6 to 6.4.

For freeze fracture electron microscopy, the nanoemulsion samples were quenched using sandwich technique in liquid nitrogen-cooled propane. Using this technique, a cooling rate of 10,000 Kelvin per second was reached avoiding ice crystals formation and artifacts possibly caused by the cryofixation process. The cryo-fixed samples were stored in liquid nitrogen for less than 2 hours before processing. The fracturing process was carried out in JEOL JED-9000 freeze-etching equipment and the exposed fracture planes were shadowed with platinum for 30 seconds at an angle of 25-35 degrees and with carbon for 35 seconds (2 kV/60-70 mA, 1×10⁻⁵ Torr). The replicas produced this way were cleaned with concentrated fuming nitric acid for 24 hours followed by repeating agitation with fresh chloroform/methanol (1:1 by volume) at least 5 times. The nanoemulsion replicas were examined using a JOEL 100CX (Peabody, Mass.) electron microscope.

Oral Absorption and Biodistribution Study

The experimental protocol involving usage of radioactive material in animals was approved by the Institutional Animal Care and Use Committee and the Office of Environmental Health and Safety at Northeastern University, Boston, Mass.

The absorption and biodistribution study of paclitaxel nanoemulsions following oral administration was conducted in 24-hour fasted female C57BL/6 mice (Charles River Laboratories Cambridge, Mass.) of 10 weeks of age (˜30 grams). A parallel study design was used with the following four groups: (1) control group (commercial paclitaxel solution for injection diluted with saline); (2) standard nanoemulsion group (paclitaxel nanoemulsions with Lipoid® E80); (3) cationic stearylamine-containing nanoemulsion (paclitaxel nanoemulsions with Lipod® E80 and stearylamine), (4) anionic; deoxycholic acid-containing nanoemulsion group (paclitaxel nanoemulsions with Lipod® E80 and deoxycholic acid). Tritiated [³H]-paclitaxel-loaded nanoemulsions, containing 2.5 μCi of labeled paclitaxel to 1050 μg of unlabeled paclitaxel, were formulated so as to contain approximately 5 μCi of radioactive dose per 5 mL of the final control and test formulations. The nanoemulsion formulations were administrated orally to conscious mice in a 1 mL volume. Blood and tissue distribution was studied at 1, 6, 12, 24 and 48 hr. Each treatment group consisted of 20 animals with n=4 for each time point.

The animals receiving control and test formulations were sacrificed after fixed time-points by cervical dislocation and the stomach, rest of the GI tract (R-GIT, which included esophagus, intestine and rectum), liver, lungs, kidney and blood (by sino-arbital vein puncture) were collected. Blood was used as such and for the tissues (stomach, R-GIT, liver, lungs and kidney), a 10% (w/v) homogenate was prepared in water and 1.0 ml each was added to a scintillation vial. All tissues and fluids (blood) were digested with Scintigest® fluid (Fisher Scientific, Pittsburgh, Pa.) using 1 mL of the Scintigest® and incubating for 2 hours at 50° C., and decolorized with hydrogen peroxide using 200 μl of 30% solution and incubated for 30 minutes at 50° C. Upon decolorization of the samples, 10 mL of the scintillation cocktail (ScintiSafe® Econo 1, Fisher Scientific) was added, and the sample was allowed to quench for 4 hours in the dark before measuring the radioactivity with a liquid scintillation analyzer (TriCarb 1600TR, Packard Instrument Co., CT). The counts-per-minute were converted into μCi using appropriate calibration curves.

Non-compartmental pharmacokinetic analysis was performed for all of the formulations. The area under the drug concentration (i.e., % radioactivity recovered per gram of tissue) versus time curve from zero to 48 hours (AUC₀₋₄₈ₕ) was calculated using the trapezoidal rule. The maximal plasma concentration of drug (Cₘₐₓ) and the time to reach maximum plasma concentration (Tₘₐₓ) were directly obtained from plasma data. The data from different formulations were compared for statistical significance by one-way analysis of variance (ANOVA). Results are considered significant at 95% confidence interval (i.e., p<0.05) and were expressed as mean±S.D.

The results of particle size analysis of paclitaxel loaded nanoemulsion formulations are depicted in Table 1.

TABLE 1
Particle size and zeta potential values of paclitaxel-loaded nanoemulsion
formulations

| | Particle Size* | | Zeta Potential |
|---|---|---|---|
| Formulation | (nm) | Polydispersity | (mV) |
| NE-Standard | 100.2 ± 9.0 | 0.115 | −29.6 ± 8.9 |
| NE-Stearylamine | 119.0 ± 9.8 | 0.106 | +34.7 ± 6.4 |
| NE-Deoxycholic acid | 90.6 ± 9.4 | 0.092 | −56.0 ± 8.0 |

*Particle size of nanoemulsions was measured using light scattering method (90Plus particle size analyzer, Brookhaven Corp., Holtsville, NY).
The formulation legends are,
NE-standard: paclitaxel loaded nanoemulsions with egg phosphatidylcholine (Lipoid ® E80; 3% w/v) as emulsifier;
NE-Stearylamine: paclitaxel loaded nanoemulsions with egg phosphatidylcholine (Lipoid ® E80; 2% w/v) and stearylamine (1% w/v) as emulsifier;
NE-Deoxycholic acid: paclitaxel loaded nanoemulsions with egg phosphatidylcholine (Lipoid ® E80; 2% w/v) and deoxycholic acid (1% w/v) as emulsifier.

Deoxycholic acid-containing nanoemulsions had the smallest mean particle size (˜90.6 nm) followed by the standard nanoemulsions (100.2 nm), and the stearylamine-containing nanoemulsions (119.0 nm). The results of freeze fracture electron microscopy studies, however, indicated that deoxycholic acid containing nanoemulsion droplets were much smaller (15 to 40 nm) with some aggregates of ˜150 nm. Stearylamine containing nanoemulsion droplets, on the other hand, were in the size range of 25 to 90 nm with aggregates between ˜100 to 300 nm. This difference in particle size analysis might be attributed to the method of analysis (Haskell, 2005). The particle size analysis using laser diffraction method (ensemble method) depends on taking a single measurement of the sample, and then the appropriate mathematical relationship is applied to extract a size population. In this method, it is thus possible that the particle size is averaged, taking into account the aggregates (as observed in FFEM) and thus represents a higher average mean value than the actual particle size. In the freeze fracture electron microscopy method (counting method), however, quantitative data is collected from individual particles, and, hence, this method presents an accurate determination of particle size distribution. From the results of these studies, it is recommended that particle size analysis of such sub-micron emulsion systems be performed by more than one method as a means of obtaining complimentary information and enabling a more comprehensive understanding of the nanoemulsion system.

The zeta potential values of the nanoemulsion formulations are also depicted in Table 1. The standard nanoemulsions and those containing deoxycholic acid formulations had a negative zeta potential values, −29.56 mv and −56.7 mV respectively, whereas the stearylamine-containing nanoemulsions had a positive zeta potential values (+34.7 mV). The electrical surface charge of the nanoemulsion droplets is produced by the ionization of the components forming the interfacial film. The present formulation is stabilized by egg phosphatidylcholine (Lipoid® E80) as the principal emulsifier. Lipoid E-80 is a mixture of phospholipids from egg yolk sources, and its major component is phosphatidylcholine which is zwitterionic and neutral over a wide pH range. The minor components of the lipid are phosphatidylserine, phosphatidic acid (PA), phosphatidylglycerol (PG), phosphatidylinositol (PI), sphingomyelin, cholesterol, and lysophosphatidylcholine. Phosphatidylserine, PA, PG, and PI are negatively charged at pH 7.0. Along with free fatty acids, these anionic fractions are probably responsible for the negative zeta potential of the standard nanoemulsion (Gaysorn et al., (1999). The negatively-charged emulsion formulation of paclitaxel was based on the use of deoxycholic acid as a co-surfactant in the formulation. The presence of deoxycholic acid was important for prolonged emulsion stability owing to its contribution to the elevated negative zeta potential of the emulsion. Un-ionized free cholic acids are known to be poor surface active agents. Nevertheless, it was preferred to prepare emulsions with deoxycholic acid instead of sodium deoxycholate as a result of better localization of the free acid at the interface of the oil/water emulsion owing to its high lipid solubility (Peimi et al., 1999). Since the pKa of deoxycholic acid is 6.5 and at the pH of the formulation (adjusted to pH 7.0), it is expected that the majority of the deoxycholic acid molecules remained in the ionized state compared to unionized fraction. The ionized fraction of the acid was probably localized in the oil/water interface of the emulsions without being excluded from the surface regions of the oil droplets. This observation is supported by the increase in negative zeta potential value of the emulsion with incorporation of deoxycholic acid in the formulation. Deoxycholic acid was also used in the nanoemulsion to potentially facilitate the oral absorption of the nanoemulsion droplets through the bile acid transporter mechanism.

Positively-charged nanoemulsions were formulated using stearylamine, a cationic lipid, as a co-surfactant. Stearylamine confers an overall positive charge to the droplets over a wide pH range (Yang et al., 2000). The positive surface potential value of the droplets of the emulsion depends mainly upon the extent of the ionization of stearylamine at the oil/water interface. Positively-charged nanoemulsions were formulated with an aim of improving the interaction of positively charged nanoemulsions with the negatively charged mucosal surface cells. It was speculated that such enhanced interaction would result is enhanced absorption of the encapsulated lipophillic drug.

Incorporation of paclitaxel did not alter the zeta potential values of the emulsion droplets as the formulations prepared without paclitaxel had similar zeta potential values. This can be explained by the lack of ionizable functional group in the paclitaxel molecule.

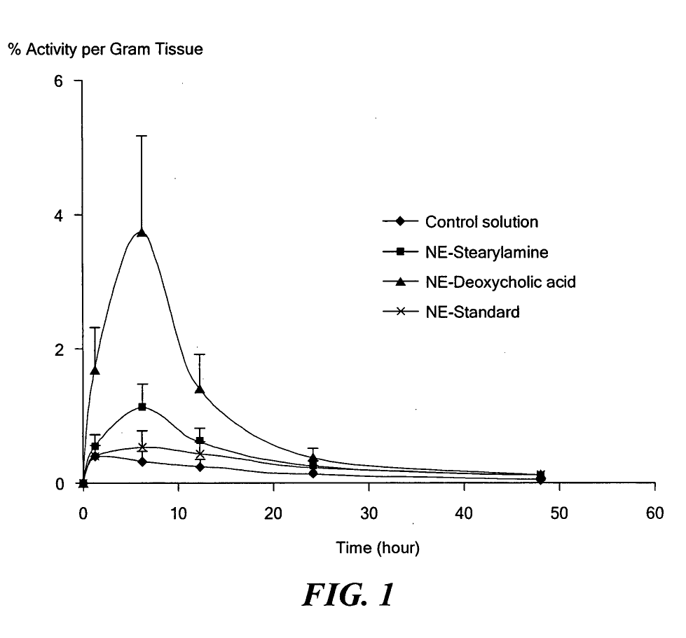

The whole blood concentration-time profile of tritiated [³H] paclitaxel in C57BL/6 mice following oral administration of nanoemulsions is shown in FIG. 1 and the summary of pharmacokinetic parameters are presented in Table 2.

TABLE 2
Summary of plasma pharmacokinetic parameters of ³H-paclitaxel
nanoemulsion formulations after oral administration in female C57BL
mice.

| | | | | NE- |
|---|---|---|---|---|
| | Control | | NE- | Deoxycholic |
| Parameter | Solution | NE-standard | Stearylamine | acid |
| AUC_{0-48 h} | 6.8 ± 1.0 | 15.9 ± 3.1^{a} | 20.6 ± 3.0^{a} | 33.5 ± 5.9^{b} |
| (% hr/g) | | | | |
| Cₘₐₓ (%/g) | 0.4 ± 0.1 | 0.51 ± 0.1 | 1.1 ± 0.40 | 3.72 ± 0.5 |
| Tₘₐₓ (hr) | 1 | 6 | 6 | 6 |
| Relative | — | 230 | 303 | 493 |
| bioavailability | | | | |
| (%) | | | | |

The formulation legends are,
Control solution: commercial paclitaxel solution for injection diluted with saline;
NE-standard: paclitaxel loaded nanoemulsions with egg phosphatidylcholine as emulsifier;
NE-stearylamine: paclitaxel loaded nanoemulsions with egg phosphatidylcholine and stearylamine as emulsifier;
NE-deoxycholic acid: paclitaxel loaded nanoemulsions with egg phosphatidylcholine and deoxycholic acid as emulsifier.
^{a}Significant at p < 0.05 as compared to the control;
^{b}Significant at p < 0.01 as compared to the control and the standard nanoemulsion formulation.

The results of the pharmacokinetic studies indicated that encapsulation of paclitaxel in nanoemulsions according to the invention did enhance the oral bioavailability of paclitaxel significantly. While not being bound by any theory, it is contemplated that the enhanced oral bioavailability of paclitaxel in nanoemulsions, as measured by the area-under-the curve (AUC), might be attributed to the solubilization of the drug in the oil droplets and/or to the presence of surfactants at the oil-water interface. Enhanced absorption of paclitaxel can also be attributed to the protection of drug from chemical as well as enzymatic degradation.

The enhancement in oral bioavailability of paclitaxel was highest with the deoxycholic acid-containing nanoemulsions, followed by the stearylamine-containing nanoemulsions and standard nanoemulsion formulations (Table 2). The enhancement in AUC of deoxycholic acid-containing nanoemulsions was nearly five-times when compared to the control aqueous paclitaxel solution and was more than two-times when compared with the standard nanoemulsion formulation. This formulation also exhibited highest paclitaxel plasma concentration (Cₘₐₓ=3.72%/g), followed by stearylamine-nanoemulsions (Cₘₐₓ=1.10%/g), the standard nanoemulsions (Cₘₐₓ=0.51%/g), and the aqueous paclitaxel solution (Cₘₐₓ=0.40%/g) Stearylamine-containing nanoemulsions were formulated so as to enhance the interaction of the positively-charged nanoemulsions with the negatively-charged mucosal cells and thus expecting higher absorption and bioavailability. However, as described earlier, the maximum enhancement in bioavailability of paclitaxel was observed with deoxycholic acid-nanoemulsions. This might be attributed to the nature of deoxycholic acid. Deoxycholic acid is a naturally occurring bile acid and because of its surface active properties, it is also used as an absorption enhancer (Sakai et al., 1999). It has been shown to enhance the oral absorption of poorly soluble drugs from the GI tract. Bile salts can decrease duodenal and jejunum brush-border membrane vesicle integrity and increase membrane fluidity which might increase the absorption of paclitaxel in the gut (Zhao et al., 1990). Moreover, deoxycholic acid is known to be a substrate for the P-glycoprotein (P-gp) efflux pump (Yang et al., 2004). As the poor oral bioavailability of the paclitaxel is attributed to its affinity for P-gp, inclusion of another P-gp substrate (deoxycholic acid) in the formulation is expected to improve its bioavailability. The results of pharmacokinetic studies also indicated the difference in Tₘₐₓ for the control solution of paclitaxel and its nanoemulsion formulations. The Tₘₐₓ for the control aqueous solution was 1 hour, whereas for nanoemulsion formulation, it was 6 hours. This difference might be attributed either to the delayed absorption of the drug in the jejunum and ileum from the oil-in-water nanoemulsions or to the differences in rate of drug diffusion to the membrane. In the case of emulsion, drug has to diffuse across oil/water interface, whereas in the case of solution, drug has to diffuse across micelle/water interface.

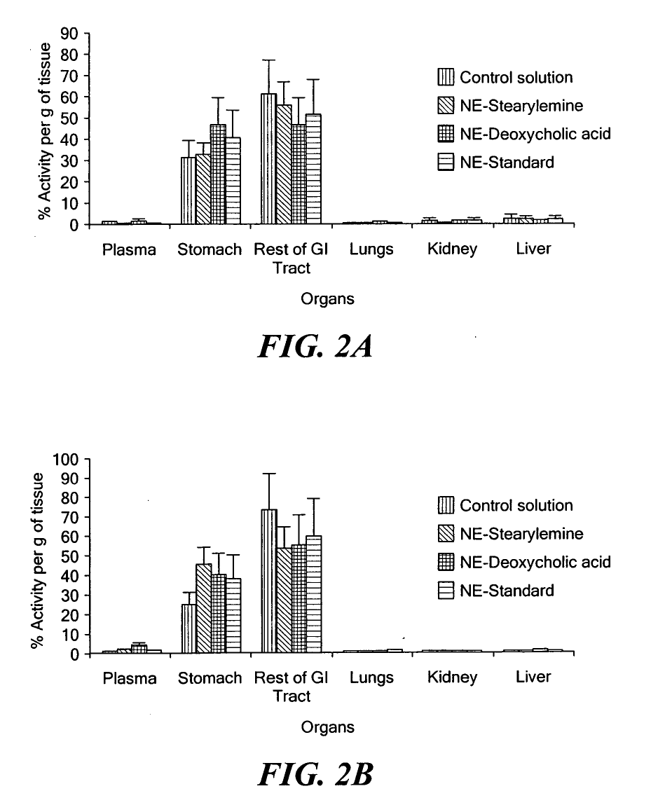

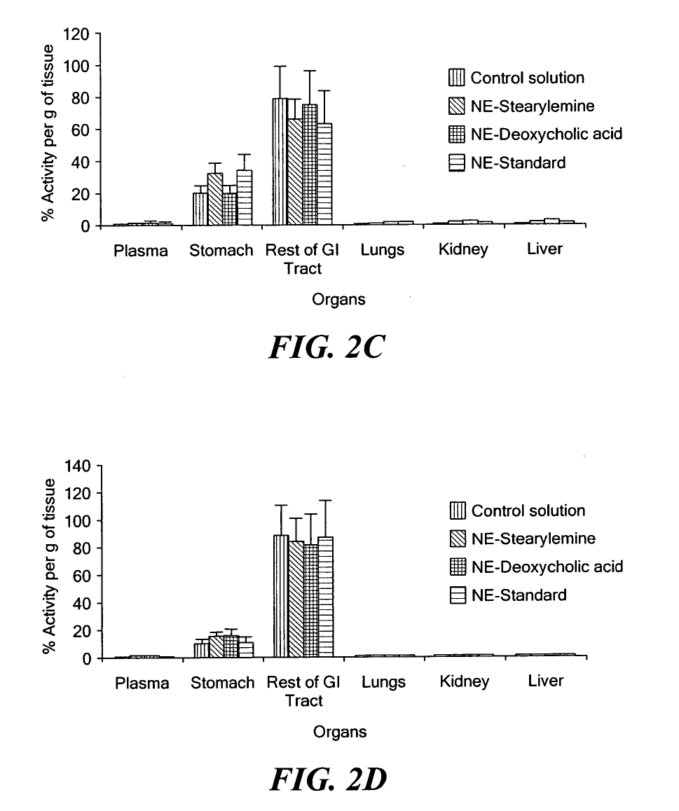

The results of tissue distribution studies, as shown in FIG. 2, indicated that the major fraction of the drug remained in the stomach and other parts of the GI tract following oral administration. The presence of paclitaxel in the liver, kidney and lungs indicate the systemic effect of the absorbed drug (Table 3 and FIG. 2). The detection of the drug in the liver indicates the possibility of a first pass effect on the orally absorbed drug.

TABLE 3
Summary of the biodistribution pattern of ³H-paclitaxel nanoemulsion formulations after
oral administration in C57BL female C57BL mice.

| Parameter | Control Solution | NE-standard | NE-Stearylamine | NE-Deoxycholic acid |
|---|---|---|---|---|
| Stomach | 722.94 ± 56.89 | 971.69 ± 78.2 | 1040.08 ± 99.23 | 941.90 ± 102.4 |
| AUC_{0-48 h} (% hr/g) | | | | |
| Rest GIT | 3954.14 ± 189.4 | 3682.75 ± 179 | 3654.49 ± 218 | 3618.81 ± 153 |
| AUC_{0-48 h} (% hr/g) | | | | |
| Liver | 2.5 ± 1.9 | 2.6 ± 2.1 | 3.0 ± 1.7 | 5.5 ± 2.9 |
| AUC_{0-48 h} (% hr/g) | | | | |
| Lungs | 1.1 ± 0.12 | 1.5 ± 1.1 | 1.1 ± 1.09 | 1.8 ± 0.89 |
| AUC_{0-48 h} (% hr/g) | | | | |
| Kidney | 2.6 ± 1.3 | 1.9 ± 1.1 | 1.9 ± 1.0 | 2.0 ± 0.95 |
| AUC_{0-48 h} (% hr/g) | | | | |

The formulation legends are,
Control solution: commercial paclitaxel solution for injection diluted with saline;
NE-standard: paclitaxel loaded nanoemulsions with egg phosphatidylcholine as emulsifier;
NE-Stearylamine: paclitaxel loaded nanoemulsions with egg phosphatidylcholine and stearylamine as emulsifier;
NE-Deoxycholic acid: paclitaxel loaded nanoemulsions with egg phosphatidylcholine and deoxycholic acid as emulsifier.

### EXAMPLE II

### Improved Oral Delivery of Saquinavir and Relationship to Specific Oil Used

Acquired immunodeficiency syndrome (AIDS) is a debilitating disease caused by the human immunodeficiency virus (HIV). More than 25 years have elapsed since the first discovery of HIV-1 as a causative agent for AIDS. There have been significant accomplishments in the past 25 years in terms of greater emphasis on disease prevention, technologies for diagnosis, and development of therapeutic strategies. At present, there are over 20 different anti-retroviral drugs under the general classes of nucleoside reverse transcriptase inhibitors (NRTI), non-nucleoside reverse transcriptase inhibitors (NNRTI), protease inhibitors (PI), and fusion inhibitors (FI) approved in the United States.

Highly-active anti-retroviral therapy (HAART) strategy involves the use of combination anti-retroviral agents for synergistic therapeutic outcomes. With adoption of HAART, the average survival of HIV/AIDS patients has increased from less than 1 year to over 10 years. Despite the success of HAART in the clinics, HIV/AIDS therapy is far from optimal. One of the major problems in the optimal treatment is the fact that the virions are able to reside in cellular and anatomical sites in the body following replication and remain viable even when there are adequate drug concentrations in the blood. Examples of cellular reservoirs include T-lymphocytes, monocytes, and macrophages, while the anatomical reservoirs include the central nervous system (CNS), lymph nodes, liver, spleen, lungs, and testes. Drug availability in the cellular and anatomical reservoirs is affected by expression of efflux transporters (e.g., P-glycoprotein), the presence of drug metabolizing enzymes (e.g., cytochrome P-450), poor permeability properties, non-targeted distribution and rapid clearance. The poor bioavailability and short residence time of anti-retroviral agents at these viral reservoir sites have a profound impact on the clinical management of the disease. The overall consequence is that upon discontinuation of therapy or when drug resistance develops, HIV is able to re-seed the systemic circulation from these reservoir sites.

Saquinavir (SQV, Invirase®), the first HIV-protease inhibitor to be marketed for treatment of HIV/AIDS, is a peptide derivative and a transition-state mimetic of the Phe-Pro peptide bond. It competitively inhibits HIV-1 and HIV-2 protease-mediated cleavage of the gag and pol polyproteins, thus preventing the post-translational processing required for virus maturation and spread Therapeutic strategy for treatment of AIDS has undergone a paradigm shift over the past decade wherein, targeted or preferential delivery of the drugs to the site of action is emerging as a novel key strategy. Preferential drug delivery may result in specific distribution at the targeted site with limited or poor distribution to the non-target sites and can potentially increase efficacy and reduce toxicity of drugs by altering the pharmacokinetics and biodistribution that restricts access of the drugs to the target and particularly to the reservoir sites. Although SQV has a very potent anti-HIV activity in vitro (IC₅₀ of 20 nM), it is currently not indicated for use as a single agent. In addition, when SQV is used in combination therapy protocols, the oral daily dose ranges from 1200 mg to 3400 mg. This is due to the fact that the oral bioavailability of SQV is only 4% from the conventional hard gelatin capsule formulation. SQV is a substrate for P-glycoprotein efflux transporter on the enterocytes and is also metabolized by the cytochrome P-450 enzymes. In addition, SQV is not adequately transported into the CNS or other anatomical reservoir sites.

In the present study, in order to enhance delivery of SQV to anatomical reservoirs, we have formulated this drug in different nanoemulsion formulations according to the invention made either with flax-seed oil or safflower oil. Flax-seed oil contains up to 57% by weight of linolenic acid, an example of an omega-3 fatty acid, and 17% by weight linoleic acid, an example of an omega-6 fatty acid. Safflower oil, on the other hand, contains up to 73% by weight linoleic acid. To examine the oral bioavailability and distribution to vital organs including the brain, SQV was incorporated in the indicated nanoemulsions and administered orally to conscious Balb/c mice. Intravenous administration was also carried out to determine the absolute bioavailability of SQV following oral administration. A control preparation of SQV was made as an aqueous suspension containing all of the other ingredients (e.g., surfactants) except the oils.

Materials and Methods

Saquinavir base was purchased from Aapin Chemicals Limited (Abingdon, United Kingdom). Tritiated [³H]-saquinavir, with an activity of 250 μCi in 250 μl ethyl alcohol was purchased from Moravek Biochemicals (Brea, Calif., USA). PUFA-containing flax-seed oil and safflower oil were kindly provided by J. Edwards International, Inc. (Boston, Mass., USA). Egg phosphatidylcholine (Lipoid® E80) was purchased from Lipoid GMBH (Ludwigshafen, Germany). Deoxycholic acid was purchased from Sigma Chemicals (St. Louis, Mo., USA). Deionized distilled water (Barnsted/Thermolyne, Dubuque, Iowa, USA) was used for the preparation of the nanoemulsions and other aqueous solutions.

Saquinavir nanoemulsions, containing a final concentration of 400 μg/mL of the therapeutic agent, were prepared by adding saquinavir solution (50% w/w stock solution in dehydrated ethanol) to 1 ml of either flax-seed oil or safflower oil. The weight ratio of radiolabeled (i.e., [³H]-saquinavir) to unlabeled drug was maintained constant at 1:50. The oil-drug mixture was stirred to homogenously distribute the drug and allow the ethanol to completely evaporate. The aqueous phase was prepared using deionized distilled water (4 mL) containing 120 mg of egg phosphatidylcholine (Lipoid E80®) and 40 mg of deoxycholic acid. The aqueous phase was also mixed to insure complete dissolution of all additives. Subsequently, both the oil phase and the aqueous phase were independently heated to 70° C. on a hot-plate.

The oil phase was gradually added to the aqueous phase with constant stirring. The resultant mixture containing both oil and aqueous phase was sonicated for 10 minutes using the Vibra Cell VC 505 probe sonicator (Sonics and Material Inc., Newtown, Conn., USA). The probe sonicator was adjusted at 21% amplitude and 50% duty cycle. The resulting stable dispersion was uniform and milky-white in color. Following sonication, the nanoemulsions were kept on a hot-plate under stirring condition and the temperature was maintained 60° C. to remove any residual ethanol and then allowed to cool to room temperature. Saquinavir-containing nanoemulsions were filtered through a 0.45 μm membrane filter and stored at 4° C. in the dark.

Since saquinavir base is not readily soluble in water, we have compared the bioavailability from the nanoemulsion preparation with an aqueous suspension formulation that was made with all of the other constituents except the oils. The aqueous suspension was prepared by mixing the ³H-labeled and unlabeled drug in ethanol with deionized distilled water containing egg phosphatidylcholine (Lipoid E80®) and deoxycholic acid at the same proportions as was used for the nanoemulsion formulations. The suspension was sonicated for 10 minutes using the Vibra Cell VC 505 probe sonicator.

The hydrodynamic oil droplet diameter in the control and saquinavir-containing nanoemulsions was measured using a light scattering method with Brookhaven Instruments Corporation's (Holtville, N.Y., USA) 90Plus ZetaPALS system. Approximately 50 μL of the nanoemulsion formulations were diluted to 5 mL using deionized distilled water in a disposable zeta cells. The observations were recorded at 900 light scattering angle and temperature was maintained at 25° C. During the measurement, average particle count rate was maintained between 50 and 500 kcps.

ZetaPALS instrument was also used for the surface charge (zeta potential) measurements of the control and saquinavir-loaded nanoemulsions. The measurements were carried out with diluted nanoemulsion formulations as described above. The refractive index was kept at 1.33 and the viscosity at 1 cps to mimic the values for pure water. Zeta potential values were determined from the electrophoretic mobility of the oil droplets using a in-built software, which uses the Helmholtz Smoluchowski equation.

The morphology of the oil droplets in the nanoemulsion formulations was visualized with TEM analysis. TEM analysis was also important in order to visualize any precipitation of the drug upon addition of the aqueous phase. Control and saquinavir-containing nanoemulsions (50 μL) were added to 200-mesh formwar-coated copper TEM sample holders (EM Sciences, Hatfield, Pa., USA). The samples were then negatively-stained with 50 μL of 1.5% (w/v) phosphotungstic acid for 10 minutes at room temperature. Excess liquid was blotted with a piece of Whatman filter paper. The TEM samples were observed with JEOL 100-X transmission electron microscope (Peabody, Mass., USA) equipped with 20 μm aperture at 67 kV. The acquired digital images were processed with Adobe Photoshop® software.

The experimental protocol involving usage of radioactive material in animals was approved by the Institutional Animal Care and Use Committee, the Radiation Safety Committee, and the Office of Environmental Health and Safety at Northeastern University (Boston, Mass.). Male Balb/c mice of approximately 10-weeks age, weighing 28-30 g, were purchased from Charles River Laboratoraties (Wilmington, Mass.). The animals were housed in a climate-controlled environment with full access to food and water. Prior to any experimentation, the animals were allowed to acclimate for at least 48 hours.

For the oral absorption and biodistribution study of saquinavir aqueous suspension (control) and the nanoemulsions formulations, the animals were fasted for 24 hours. They were then randomly divided into three groups to receive [³H]-saquinavir in aqueous suspension, flax-seed oil nanoemulsion, or safflower oil nanoemulsion formulations. Each conscious animal was administered with 0.5 mL (200 μg of saquinavir) of the control aqueous suspension or nanoemulsion formulations containing 1 μCi of radioactivity by oral gavage. After 1, 2, 4, 6, 8, 12, and 24 hours post-administration, a group of 4 animals per time point, lightly anesthetized with isoflurane, were sacrificed by cervical decapitation.

In a separate series of experiments, the [³H]-saquinavir in aqueous suspension and nanoemulsion formulations were also administered intravenously via the tail vein in isoflurane-anasthesized male Balb/c mice. For these studies, the formulations were made such 200 μg of total saquinavir and 1 μC of radioactivity was incorporated in 100 μL of the injectable aqueous suspension and nanoemulsions. At specific time points, a group of 4 anesthetized mice were sacrificed by cervical decapitation.

Following oral and intravenous drug administration, blood was rapidly collected by cardiac puncture and placed with anticoagulants. Brain, lung, heart, liver, spleen, kidneys, stomach, and intestine were collected and processed for analysis of saquinavir concentrations.

Radioactivity in the blood and isolated tissues was used to determine the concentration of saquinavir following oral and intravenous administration. The harvested tissues were weighed and rapidly homogenized using a Fisher PowerGen-125 homogenizer to prepare a 10% (w/v) in deionized distilled water. One-mL of the tissue homogenate was added to a scintillation vial. Blood and tissue homogenates were then digested with 1 mL of Scintigest® solution (Fisher Scientific, Pittsburgh, Pa., USA) and incubated for 2 hours at 50° C. The samples were then decolorized with 200 μL of 30% (v/v) hydrogen peroxide by incubating at 50° C. for an additional 30 minutes. To the decolorized samples, 10 mL of ScintiSafe® Econo-1 scintillation cocktail ((Fisher Scientific) was added and they were allowed to quench for 4 hours in the dark. Radioactivity analysis was performed with a Packard Instrument's Tri-Carb 1600TR liquid scintillation analyzer (???,CT) after appropriate calibration with tritium standards. The counts-per-minute (CPM) values were converted into μCi of radioactivity per gram of each tissue or fluid using calibration curves.

Non-compartmental pharmacokinetic analysis of saquinavir following oral and intravenous administration in blood and various harvested tissues was performed with WinNonlin, version 5.0 software package (Pharsight Corporation, Mountain View, Calif., USA) Pharmacokinetic parameters such as the maximum plasma concentration (Cₘₐₓ), time to reach maximum concentration (Tₘₐₓ), volume of distribution (V_{d}), total body clearance (C_{L}), half-life (t_{1/2}), mean residence time (MRT), and area-under-the-curves (AUC_{0→24} and AUC_{0→∞}) were calculated. Absolute oral bioavailability (F) of saquinavir in the aqueous suspension and nanoemulsion formulations was determined according to the following equation:

$$F = \frac{AUC ( oral ) * Dose ( i . v . )}{AUC ( oral ) * Dose ( oral )}$$

All the values are reported as mean±SEM and the difference between the groups were tested using student's t-test and, with more than two groups, ANOVA was used to compare results. Experimental results were considered statistically significant at 95% confidence (i.e., p<0.05).

Oral Bioavailability and Brain Uptake Study

SQV-containing nanoemulsions according to the invention were prepared by the ultra sonication method as described above. As shown in Table 4, the average oil droplet diameters (particle size) of the SQV-containing nanoemulsions were in the range of 100-200 nm. Blank nanoemulsions, prepared in the absence of the drug, also had similar oil droplet size of about 200 nm in diameter. Ultrasound was also used here to reduce the particle size of solid drug precipitate in the control aqueous suspension to approximately 300 nm in diameter. In addition, the change in the oil droplet particle size was also examined as a function of various centrifugation cycles as an accelerated stability indicating system. There was no change in particle size of the oil droplets in these nanoemulsions upon centrifugation at up to 2,000 rpm for 20 minutes as well as on storage at 4° C. in the dark for up to two months.

TABLE 4
Particle Size and Surface Charge Measurements of the Control Suspension
and Nanoemulsions Formulations of Saquinavir^{a}

| | | Zeta Potential |
|---|---|---|
| Formulations | Particle Size (nm) | (mV) |
| Control Aqueous Suspension | 311.2 ± 17.4^{b} | −9.42 ± 4.28 |
| Blank Flax-Seed Oil | 176.6 ± 18.2 | −39.56 ± 3.67 |
| Nanoemulsion | | |
| Saquinavir-Containing Flax- | 218.0 ± 13.9 | −43.28 ± 3.79 |
| Seed Oil Nanoemulsion | | |
| Blank Safflower Oil | 217.4 ± 11.6 | −45.62 ± 4.29 |
| Nanoemulsion | | |
| Saquinavir-Containing | 140.0 ± 12.6 | −49.55 ± 5.02 |
| Safflower Oil Nanoemulsion | | |

^{a}Particle size and surface charge measurements of the aqueous suspension and nanoemulsion formulations using the ZetaPALS instrument.
^{b}Mean ± S.E. (n = 3).

Table 4 also shows the surface charge of the various types of nanoemulsions with and without added SQV. Both flax-seed and safflower oil nanoemulsions showed net negative zeta potential values of approximately −40 mV. In this study, egg phosphatidylcholine (Lipoid® E-80) was used as the primary emulsifier for stabilization of the oil droplets. Literature evidence suggests that Lipoid® E-80 is a mixture of phospholipids from egg yolk sources with the major constituent being phosphatidylcholine. Lipoid® 80 also contains a small fraction of zwitterionic and neutral phospholipids such as phosphatidylserine, phosphatidic acid, phosphatidylglycerol, and phosphatidylinositol that would contribute a net negative charge at neutral pH. More importantly, deoxycholic acid was included as a co-surfactant in the nanoemulsion preparation in order to enhance oral absorption. With a pKa of 6.5, a significant fraction of the deoxycholic acid molecules would be ionized at neutral pH. Adsorbed deoxycholic acid at the oil-water interface would clearly impart a significant net negative charge to the nanoemulsions. Inclusion of SQV did not alter the surface charge of the nanoemulsion.

In order to observe the physical properties of the oil droplets in the nanoemulsions, TEM analysis was carried out with negatively stained samples. Phosphotungstic acid-stained oil droplets were clearly visible, and the droplet size correlated well with the results from particle size analysis using ZetaPALS light scattering instrument. In addition, the morphology of the droplet was spherical, and there was no evidence of SQV precipitation in either the oil phase or the aqueous phase with the concentrations that were incorporated in both flax-seed and safflower oil nanoemulsions.

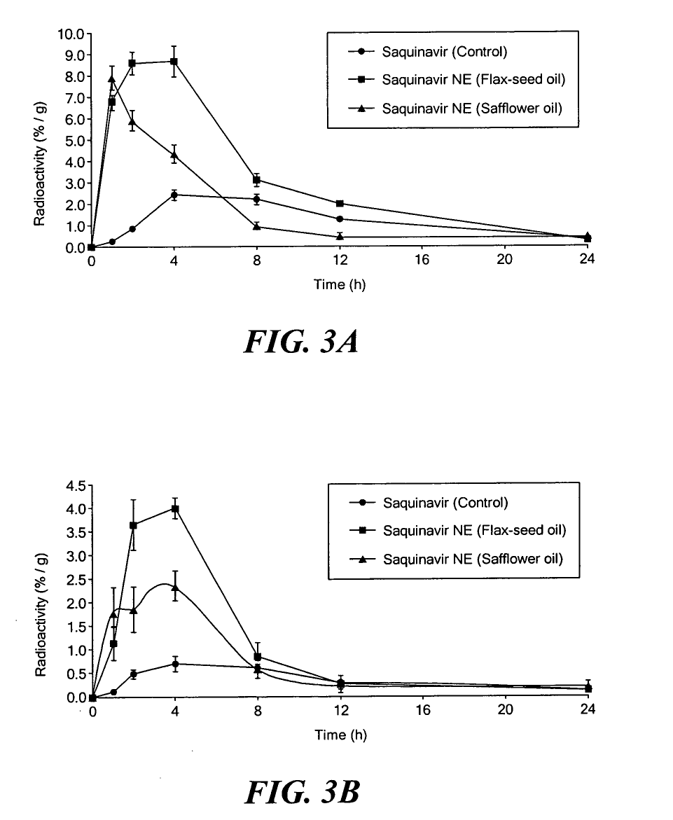

In order to evaluate the enhanced bioavailability and distribution pattern in the body following oral administration, control aqueous suspension and the nanoemulsion formulations containing [³H]-SQV were administered to conscious male Balb/c mice. The mice were dosed with 0.50 mL of the control aqueous suspension or nanoemulsion formulations by oral gavage. The administered dose contained 1 μCi radioactivity as tritiated [³H]-saquinavir. The graphs presented in FIGS. 3A and 3B give the plasma and brain concentration versus time profile of SQV following oral administration in aqueous suspension, flax-seed oil nanoemulsion, and safflower oil nanoemulsion. These results clearly show that there were higher plasma and brain SQV concentrations when administered orally in the nanoemulsions as compared to the aqueous suspension. Flax-seed oil nanoemulsion was more effective in enhancing oral absorption and availability of SQV in the plasma as well as in the brain. The average plasma concentrations of 8.7% dose/gram and 5.9% dose/gram of SQV were observed after 2 hours following oral administration in flax-seed oil and safflower oil nanoemulsions, respectively. In contrast, the plasma concentration of SQV from the aqueous suspension after 2 hours was only 0.82% dose/gram (FIG. 3A). In addition, brain concentrations of SQV were also significantly higher (p<0.05) when administered with flax-seed and safflower oil nanoemulsions as compared to the aqueous suspension. After 2 hours of oral administration, the average SQV concentrations in the brain were 3.7% dose/gram with flax-seed oil nanoemulsion, 1.9% dose/gram with safflower oil nanoemulsion, and 0.49% dose/gram with the aqueous suspension formulation (FIG. 3B). Plasma and brain SQV concentrations when administered with the nanoemulsions remained higher than with aqueous suspension for up to 8 hours.

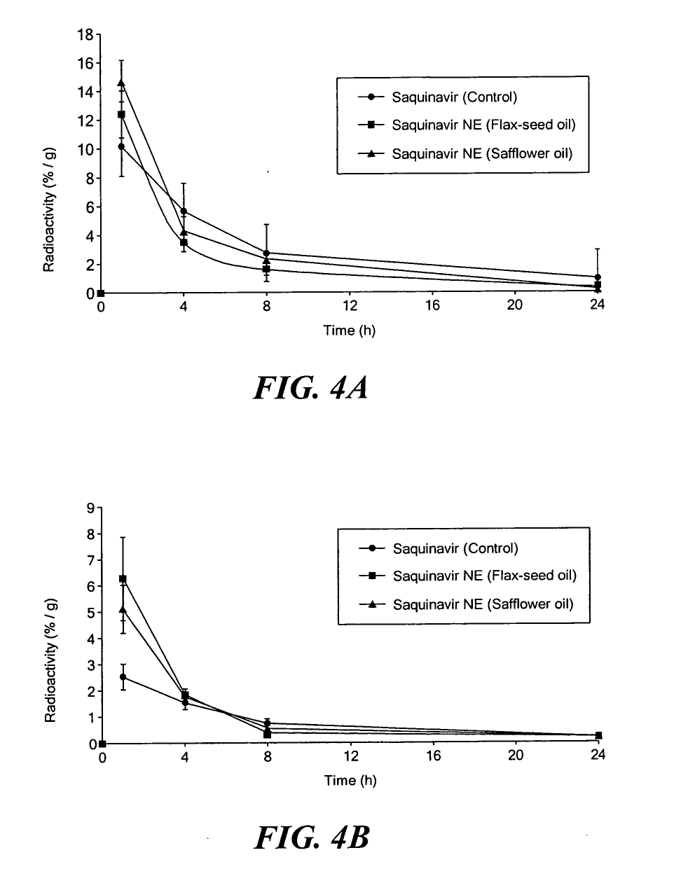

The results of i.v. SQV administration in the aqueous suspension and in the nanoemulsion formulations, and, thus, the effectiveness of oral administration relative to i.v. delivery, are given in FIGS. 4A and 4B. The Balb/c mice were dosed with 0.10 mL of the control aqueous suspension or nanoemulsion formulations by the intravenous tail vein injection. Each administered dose contained 1 μCi radioactivity as tritiated [³H]-saquinavir. At 1 hour post i.v. administration, SQV concentration in the plasma was 12.4% dose/gram with flax-seed oil nanoemulsion, 14.7% dose/gram with safflower oil nanoemulsion, and 10.2% dose/gram with the aqueous suspension formulation (FIG. 4A). However, the brain SQV concentrations were significantly higher (p <0.05) when administered i.v. in the nanoemulsions as compared to the aqueous suspension. Brain SQV concentration after 1 hour, for instance, was 6.3% dose/gram with flax-seed oil nanoemulsion, 5.1% dose/gram with the safflower oil nanoemulsion, and 2.6% dose/gram with the aqueous suspension (FIG. 4B).

Non-compartmental pharmacokinetic analysis was carried to determine the plasma and brain parameters following oral and intravenous administration. In Table 5, the results of pharmacokinetic analysis in the plasma following oral and i.v. administration of SQV in aqueous suspension and in the nanoemulsion formulations are shown. The maximum plasma concentration (Cₘₐₓ), time-to-reach maximum plasma concentration (Tₘₐₓ), the half-life (T_{1/2}), and area-under-the-curve (AUC_{0→∞}) values were used to compare the differences between the three formulations.

TABLE 5
Pharmacokinetics Parameters Upon Oral and Intravenous Administration of the Control Suspension and Nanoemulsion
Formulations of [³H]-Saquinavir in the Plasma Compartment in Balb/c Mice^{a}

| | Saquinavir Control | | | | Saquinavir in Flax-Seed Oil | | | |
|---|---|---|---|---|---|---|---|---|
| | Suspension | | | | Nanoemulsion | | | |
| Route of | Cₘₐₓ | Tₘₐₓ | T_{1/2} | AUC_{0→∞} | Cₘₐₓ | Tₘₐₓ | T_{1/2} | AUC_{0→∞} |
| Administration | (% ID/g) | (h) | (h) | h(% ID/g) | (% ID/g) | (h) | (h) | h(% ID/g) |
| Oral | 2.42 ± 0.34 | 4.00 ± 0.20 | 5.93 ± 0.64 | 32.00 ± 4.73 | 8.70 ± 0.46^{b,c} | 4.00 ± 0.55 | 3.86 ± 0.44^{b,c} | 76.55 ± 6.82^{b,c} |
| Intravenous | 10.22 ± 1.62 | 1.00 ± 0.05 | 5.57 ± 0.74 | 70.55 ± 5.53 | 12.38 ± 1.27^{b,c} | 1.00 ± 0.10 | 6.14 ± 0.78^{b,c} | 59.34 ± 4.68^{b,c} |

| | Saquinavir in Safflower Oil | | | |
|---|---|---|---|---|
| | Nanoemulsion | | | |
| Route of | Cₘₐₓ | Tₘₐₓ | T_{1/2} | AUC_{0→∞} |
| Administration | (% ID/g) | (h) | (h) | h(% ID/g) |
| Oral | 7.93 ± 0.59^{b} | 1.00 ± 0.20 | 4.99 ± 0.28^{b} | 42.30 ± 5.45^{b} |
| Intravenous | 14.71 ± 1.16^{b} | 1.00 ± 0.05 | 4.63 ± 0.36^{b} | 80.64 ± 6.83^{b} |

^{a}The mice were administered with [³H]-saquinavir formulations (1 μCi) and the radioactivity were measured as percent of administered dose per gram of fluid or tissue. Each value is a mean ± S.E. of four independent experiments.
^{b}Difference was found to be significant (p < 0.05) as compared with saquinavir control suspension.
^{c}Difference was found to be significant (p < 0.05) as compared with saquinavir nanoemulsions in safflower oil.

Following oral administration, the average Cₘₐₓ and AUC values were 8.7% dose/gram 76.6 h(% dose/gram) with the flax-seed oil nanoemulsion and 7.9% dose/gram and 42.3 h(% dose/gram) with the safflower oil nanoemulsion, respectively. In contrast, the Cₘₐₓ and AUC values following oral administration with SQV suspension was 2.4% dose/gram and 32.0 h(% dose/gram), respectively. Upon i.v. administration, the plasma concentration versus time AUC values were 59.3 h(% dose/gram) with flax-seed oil nanoemulsion, 80.6 with the safflower oil nanoemulsion, and 70.6 h(% dose/gram) with SQV aqueous suspension.

TABLE 6
Pharmacokinetics Parameters Upon Oral and Intravenous Administration of the Control Suspension and Nanoemulsion
Formulations of [³H]-Saquinavir in the Brain Compartment in Balb/c Mice^{a}

| | Saquinavir Control | | | | Saquinavir in Flax-Seed Oil | | | |
|---|---|---|---|---|---|---|---|---|
| | Suspension | | | | Nanoemulsion | | | |
| Route of | Cₘₐₓ | Tₘₐₓ | T_{1/2} | AUC_{0→∞} | Cₘₐₓ | Tₘₐₓ | T_{1/2} | AUC_{0→∞} |
| Administration | (% ID/g) | (h) | (h) | h(% ID/g) | (% ID/g) | (h) | (h) | h(% ID/g) |
| Oral | 0.73 ± 0.09 | 4.00 ± 0.35 | 9.92 ± 0.47 | 11.80 ± 2.03 | 4.02 ± 0.27^{b,c} | 4.00 ± 0.35 | 4.48 ± 0.35^{b,c} | 25.90 ± 1.98^{b,c} |
| Intravenous | 2.50 ± 0.31 | 1.00 ± 0.10 | 5.15 ± 0.73 | 17.12 ± 1.84 | 6.26 ± 0.45^{b,c} | 1.00 ± 0.10 | 4.76 ± 0.61^{b} | 23.78 ± 2.45^{b} |

| | Saquinavir in Safflower Oil | | | |
|---|---|---|---|---|
| | Nanoemulsion | | | |
| Route of | Cₘₐₓ | Tₘₐₓ | T_{1/2} | AUC_{0→∞} |
| Administration | (% ID/g) | (h) | (h) | h(% ID/g) |
| Oral | 2.38 ± 0.55^{b} | 4.00 ± 0.30 | 6.45 ± 0.84^{b} | 19.01 ± 1.74^{b} |
| Intravenous | 5.12 ± 0.48^{b} | 1.00 ± 0.10 | 4.43 ± 0.33^{b} | 29.00 ± 3.54^{b} |

^{a}The mice were administered with [³H]-saquinavir formulations (1 μCi) and the radioactivity were measured as percent of administered dose per gram of fluid or tissue. Each value is a mean ± S.E. of four independent experiments.
^{b}Difference was found to be significant (p < 0.05) as compared with saquinavir control suspension.
^{c}Difference was found to be significant (p < 0.05) as compared with saquinavir nanoemulsions in safflower oil.

Table 6 shows the pharmacokinetic parameters of SQV following administration in the aqueous suspension and the nanoemulsion formulations in the brain. Following oral administration, the average Cₘₐₓ and AUC values in the brain were 4.0% dose/gram and 26.0 h(% dose/gram) when SQV was administered in flax-seed oil nanoemulsion, 2.4% dose/gram and 19.0 h(% dose/gram) when administered in safflower oil nanoemulsion. In contrast, oral administration of SQV in the aqueous suspension resulted in the average Cₘₐₓ and AUC values of 0.7% dose/gram and 11.8 h(% dose/gram), respectively. Upon i.v. administration, the SQV Cₘₐₓ and AUC values were also affected by the delivery system. The average Cₘₐₓ and AUC values in the brain were 6.3% dose/gram and 23.8 h(% dose/gram) when SQV was administered in flax-seed oil nanoemulsion, 5.1% dose/gram and 29.0 h(% dose/gram) when administered in safflower oil nanoemulsion. When SQV was administered i.v. in the aqueous suspension, the average Cₘₐₓ and AUC values were 2.5% dose/gram and 17.1 h(% dose/gram), respectively.

To compare the availability of SQV in the brain following oral and i.v. administration, the ratio of concentrations in the brain at different time points from 1 hour to 24 hours post-administration were determined. As shown in Table 7, the brain oral/i.v. concentration ratios with flax-seed oil nanoemulsion were 2.2 and 2.9 at 4 hours and 8 hours, respectively. With safflower oil, the ratios were 0.70 and 1.2 at hours and 8 hours, respectively. Aqueous suspension was not very effective in brain delivery following oral administration and, as such, the concentration ratios were 0.49 and 0.91 at 4 hours and 8 hours, respectively.

TABLE 7
Ratio of Brain Concentrations of [³H]-Saquinavir Following Oral and Intravenous Administration in
the Control Suspension and Nanoemulsion Formulations in Balb/c Mice^{a}

| Formulation and Route | | | | | |
|---|---|---|---|---|---|
| of Administration | Compartments | 1 h | 4 h | 8 h | 24 h |
| Control Suspension | Brain Oral/Brain Intravenous | 0.05 ± 0.02 | 0.49 ± 0.08 | 0.91 ± 0.06 | 1.36 ± 0.21 |
| Saquinavir Nanoemulsion | Brain Oral/Brain Intravenous | 0.19 ± 0.05^{b,c} | 2.22 ± 0.07^{b,c} | 2.90 ± 0.11^{b,c} | 0.93 ± 0.11^{b} |
| in Flax-Seed Oil | | | | | |
| Saquinavir Nanoemulsion | Brain Oral/Brain Intravenous | 0.35 ± 0.09^{b} | 0.70 ± 0.12^{b} | 1.18 ± 0.16^{b} | 1.54 ± 0.08^{b} |
| in Safflower Oil | | | | | |

^{a}The mice were administered with [³H]-saquinavir formulations (1 μCi) and the radioactivity were measured as percent of administered dose per gram of fluid or tissue. Each value is a mean ± S.E. of four independent experiments.
^{b}Difference was found to be significant (p < 0.05) as compared with saquinavir control suspension.
^{c}Difference was found to be significant (p < 0.05) as compared with saquinavir nanoemulsions in safflower oil.

TABLE 8
Relative Bioavailabilities of [³H]-Saquinavir Upon Administration in Control Suspension and
Nanoemulsion Formulations to Balb/c Mice^{a}

| | Bioavailability | Bioavailability | Bioavailability | Bioavailability |
|---|---|---|---|---|
| | Following Intravenous | Following Oral | Following Intravenous | Following Oral |
| | Administration in Blood | Administration in Blood | Administration in Brain | Administration in Brain |
| Formulation | (%) | (%) | (%) | (%) |
| Control Suspension | 100 ± 5.0 | 45.0 ± 3.5 | 100 ± 3.0 | 69.0 ± 3.0 |
| Saquinavir | 84.0 ± 3.0^{b} | 109 ± 4.0^{b,c} | 139 ± 3.5.0^{b} | 151 ± 4.0^{b,c} |
| Nanoemulsion | | | | |
| in Flax-seed Oil | | | | |
| Saquinavir | 114 ± 4.5^{b} | 60.0 ± 2.0^{b} | 169 ± 3.0^{b} | 111 ± 3.0^{b} |
| Nanoemulsion | | | | |
| in Safflower Oil | | | | |

^{a}The mice were administered with [³H]-saquinavir formulations (1 μCi) and the radioactivity were measured as percent of administered dose per gram of fluid or tissue. Each value is a mean ± S.E. of four independent experiments.
^{b}Difference was found to be significant (p < 0.05) as compared with saquinavir control suspension.
^{c}Difference was found to be significant (p < 0.05) as compared with saquinavir nanoemulsions in safflower oil.

The relative bioavailability values of SQV in the plasma and brain following oral and i.v. administration in the aqueous suspension and the nanoemulsion formulations are shown in Table 8.

The bioavailability values were normalized based on the AUC values of the aqueous suspension formulation administered i.v. From the results, it is clear that the SQV formulation in the flax-seed oil nanoemulsion afforded significant improvement in the bioavailability of the drug in plasma and brain upon oral administration. As compared to the average availability of 45% of the drug in aqueous suspension upon oral administration, the flax-seed oil nanoemulsion and safflower oil nanoemulsion afforded the average relative bioavalabilities of 109% and 60%, respectively. Most importantly, oral administration of SQV in flax-seed (151%) and safflower oil (111%) nanoemulsions provided significant improvement (p<0.05) in average availability to the brain as compared to the aqueous suspension (69%). Upon i.v. administration, the average relative bioavalabilities of SQV in the plasma with flax-seed oil and safflower oil nanoemulsions were 84% and 114%, respectively, as compared to the aqueous suspension formulation. Additionally, following i.v. administration, flax-seed oil and safflower oil nanoemulsions provided average relative bioavalabilities of 139% and 169%, respectively, in the brain.

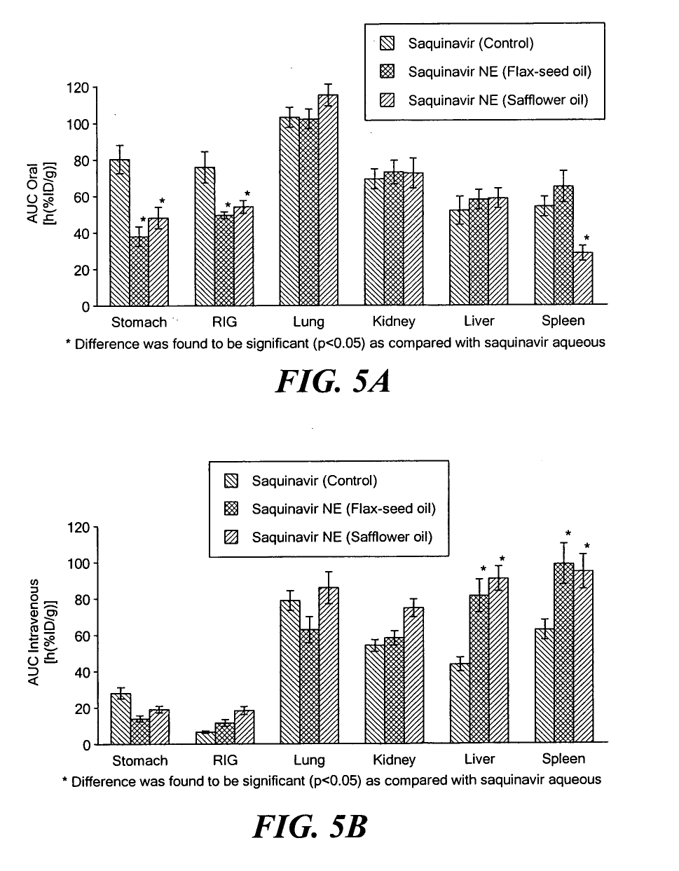

In addition to plasma and brain, the SQV concentrations versus time in the gastrointestinal tract and major perfusing organs were also examined. Each mouse received 0.50 mL by oral gavage or 0.10 mL by intravenous tail vein injection of the formulation containing 1 μCi of radioactivity as tritiated [³H]-saquinavir either in aqueous suspension or nanoemulsion formulations. FIGS. 5A and 5B show the AUC values of SQV concentrations versus time profile in the stomach, rest of the gastrointestinal tract (RIG), lung, kidneys, liver, and spleen. Following oral administration, there was a significant difference (p<0.05) in the AUC values in the stomach and RIG following administration in aqueous suspension and in the nanoemulsion formulations (FIG. 5A). The average stomach AUC values were 39.8 h(% dose/gram) for flax-seed oil nanoemulsion, 49.2 h(% dose/gram) with the safflower oil nanoemulsion, and 80.0 h(% dose/gram) with the aqueous suspension formulation. In the RIG, the average AUC values were 45.3 h(% dose/gram) for flax-seed oil nanoemulsion, 55.3 h(% dose/gram) with the safflower oil nanoemulsion, and 78.5 h(% dose/gram) with the aqueous suspension formulation. The AUC values in the lung, kidneys, and liver were not statistically different when SQV was administered orally in the aqueous suspension as compared to the nanoemulsions.

Upon i.v. administration, the average AUC values in liver and spleen were significantly higher (p<0.05) with the nanoemulsion formulation as compared to the aqueous suspension formulation (FIG. 5B). The average AUC values of SQV concentrations versus time profiles in the liver were 40 h(% dose/gram), 79.5 h(% dose/gram), and 91.0 h(% dose/gram) administered i.v. in aqueous suspension, flax-seed oil nanoemulsion, and safflower oil nanoemulsion, respectively. Additionally, in the spleen, the average AUC values were 62.4 h(% dose/gram) when administered in aqueous suspension, 101.8 h(% dose/gram) when administered in flax-seed oil nanoemulsion, and 95.4 h(% dose/gram) when administered in safflower oil nanoemulsion.

The results of this study show that nanoemulsion during delivery systems according to the invention may be very promising for HIV/AIDS therapy, in particular, in reducing the viral load at important anatomic reservoir sites.

### EXAMPLE, III

### Multifunctional Nanoemulsions

Nanotechnology has exploded into the forefront of medical research because of its potential for use in molecular imaging in addition to targeted drug delivery. Nanoparticles and nano-assemblies have been shown to direct drugs to specific body tissues, such as solid tumors or the brain, and to ameliorate adverse side effects from free-flowing toxins (Brannon-Peppas et al., 2004; Schroeder et al., 1998; Schroeder et al., 1998 and Gulyaev et al., 1999). Additionally, nanoparticles are small enough to travel into minute body regions and, when coupled with paramagnetic elements such as gadolinium ions (Gd³⁺), can enhance tissue contrast in magnetic resonance imaging (MRI) (Torchilin et al., 2002; Reynolds et al., 2000). One of the central challenges in cancer diagnosis and therapy is the ability to visualize the tumor boundary and target drugs to that area. Employing target-specific drug delivery would mitigate drug side effects, a serious threat that constitutes one of the leading causes of death from debilitating diseases such as cancer (Lazarou et al., 1998). Furthermore, once the drug has reached the target site, it is important to be able to determine its efficacy using an in vivo imaging modality such as MRI. Image-guided therapy couples drug delivery with tissue imaging to allow clinicians to specifically deliver therapeutics, while simultaneously localizing the drugs and visualizing their physiological effects.

In addition to their drug delivery capabilities, nanoparticulate systems can also act as MRI contrast agents to enhance tissue image resolution (Torchilin, 2002). Contrast agents such as Gd³⁺ have unpaired electrons that interact with surrounding water molecules to decrease their proton spin time, also referred to as T₁ (Gimi et al., 2005). Relaxation time is defined as the period it takes for a proton to return to its equilibrium position following a magnetization pulse. Nuclear magnetic resonance (NMR) and MRI can measure T₁ by creating a magnetic field that reverses the sample's magnetization, then recording the time required for the spin directions to realign in their equilibrium positions again (Torchilin, 2000). The decreased T₁ relaxation time of the target tissue allows an MRI machine to better distinguish between it and its surrounding aqueous environment (Lanza et al., 2003). Since it is impossible to dose patients with paramagnetic ions directly, given their inherent toxicity, current MRI contrast technology entails sequestering the metal using an organic chelate (Leach, 2001). The chelating ligand acts as a carrier that provides safe transport into and out of body. With regard to MRI, the first contrast agents to be used routinely were Gd³⁺ chelates such as Gd³⁺-DTPA and Gd³⁺-DOTA (Reynolds et al., 2000). Apart from commercially available Gd³⁺-DTPA solutions (Magnevist), Berlex Labs, Wayne, N.J.), efforts have been directed to engineering paramagnetic nano/microparticulate systems such as liposomes, micelles, or dendrimer-based metal chelates (Torchilin, 2002; Torchilin, 2000).

The nanoemulsion delivery system according to the invention can serve as a new class of Gd³⁺ chelated 50-100 nm multifunctional nanoemulsions that not only can exhibit MR contrast but can also carry encapsulated drugs to the target tissue for successful image-guided therapy. To examine the MRI contrast potential of these nanoemulsions, in vitro studies were conducted using NMR and MRI, while cell uptake and trafficking as well as cytotoxicity studies were conducted to examine the drug delivery potential of the multifunctional system.

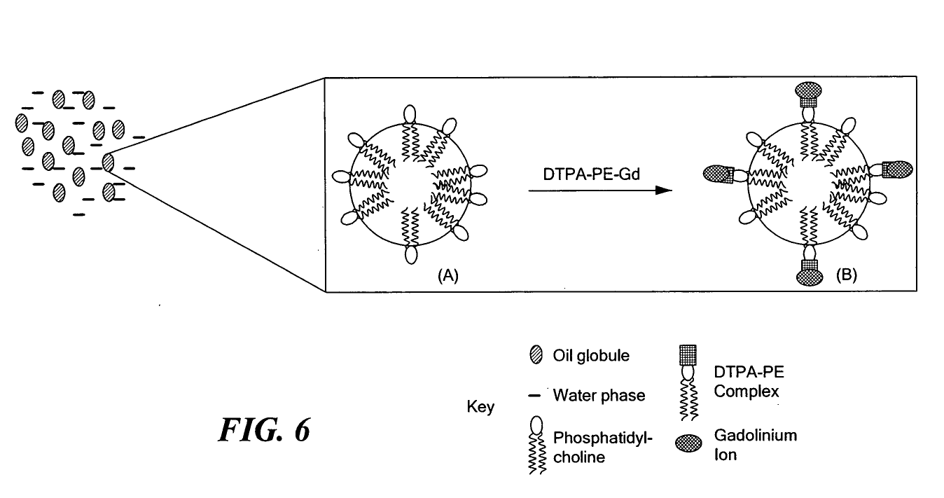

FIG. 6 is a schematic representation of a multifunctional nanoemulsion and also of the anatomy of one nanoparticle (e.g., oil globule or nanodroplet) in the nanoemulsion complexed with DTPA-PE-Gd⁺³; Schematic A (before complexing) shows the surface arrangement of phosphatidylcholine molecules with the lipophilic “tail” embedded within the oily droplet while the polar “head” groups are positioned at the interface of the oil and water phases; Schematic B (after complexing) shows the association of DTPA-PE-Gd molecules with the oily droplet in an analogous manner to phosphatidylcholine, with the DTPA-gadolinium molecules also being positioned at the interface of the oil and water phases.

Preparation of the Nanoemulsions

Nanoemulsions were prepared by the sonication method. To 2 ml of deionized water, 60 mg of egg phosphatidylcholine (Lipoid® E80, Lipoid GMBH, Ludwigshfaen, Germany) and ˜56 μl of glycerol were added; the mixture was stirred at 5,000 rpm for 10 minutes using Fisher PowerGen-125 homogenizer. Separately, a 0.5 ml aliquot of Siberian Tiger Natural's pine-nut oil (Cabot, Vt.), which has at least 20% by weight of GLA, was taken. The two phases were then heated separately on a hot plate at 70° C. for 3-5 minutes. The oil phase was added to the aqueous solution and the mixture was sonicated at 21% amplitude and 50% duty cycle with a Vibra Cell® VX-505 instrument (Sonics and Materials, Newtown, Conn.) for 10 minutes resulting in the formation of the nanoemulsion. The surface charge of the nanoemulsion was also modified by addition of either positively-charged stearylamine or negatively-charged deoxycholic acid.

Preparation of Diethylenetriaminepentaacetic Acid (DTPA)-Phosphatidylethanolamine (PE) Complex

The DTPA-PE ligand was synthesized using the method as reported earlier (Levchenko et al., 2002). Briefly, 100 mg of egg phosphatidylethanolamine (PE) was dissolved in 4 ml of chloroform. To this, 30 μl of triethylamine solution was added. This solution was then added drop-wise to the DTPA anhydride solution (400 mg in 20 ml of dimethylsulfoxide) and the mixture was stirred for 3 hours. The solution was then dialyzed against deionized distilled water at 4° C. using a DTPA-saturated dialysis bag (Spectra/Por®2, MWCO 12,000, Spectrum Laboratories Inc., Rancho Dominguez, Calif.) for 48 hours. Chloroform residue was removed by a rotary evaporator followed by freeze-drying of the sample. The purity of the complex was determined by thin layer chromatography using a mobile phase of chloroform:methanol:water at 65:25:4 (v/v) ratio and ninhydrin as visualizing reagent. The R_{f} value of 0.4 confirmed the formation of the DTPA-PE complex (Grant et al., 1989).

The nanoemulsions were made by conjugating the surfactant with DTPA-PE complex. The conjugated PE molecules can then form chelating sites for Gd³⁺ ions. In order to anchor Gd³⁺ ions, GdCl₃ was dissolved in water to yield 20 mg/ml of solution. Addition of DTPA-PE and Gd³⁺ to the nanoemulsions resulted in Gd³⁺ spontaneously chelating to the DTPA, due to their high affinity for one other, and the PE being adsorbing at the interface of the oil and water phase. DTPA-PE provides the correct anchoring position for Gd³⁺ ions on the outer surface of the oil droplets.

Characterization of the Nanoemulsions

The mean particle size of the oil droplets in the nanoemulsion was measured using a Brookhaven Instrument's ZetaPALS® 90Plus (Holtsville, N.Y.). The colloidal dispersion was diluted with deionized distilled water and the particle size analysis was carried out at a 900 scattering angle and temperature of 25° C. Approximately, 50 μl of the nanoemulsion was diluted to 1 ml before passing it through the particle sizer and the average count rate was kept between 50-500 kcps.

Zeta potential (ξ) measurements of the nanoemulsions in deionized water were performed using the ZetaPALS instrument as well. The zeta potential values were measured at the default parameters of the dielectric constant, refractive index, and viscosity of water, and were calculated based on the electrophoretic mobility. Approximately, 1.0 ml of diluted nanoemulsion was connected to an electrode at 4 volts and 2 Hz field frequency for these measurements.

NMR and MRI Experiments

Both nuclear magnetic resonance (NMR) and magnetic resonance imaging (MRI) were used to determine the T₁ relaxation times of the Gd³⁺-containing nanoemulsions. For NMR, 10 μl of nanoemulsion was loaded into glass capillaries and subjected to 500 MHz of magnetization in an 11.7 Tesla field on an Oxford AS500 (Austin, Tex.) NMR instrument. A magnetic pulse was sent through the machine, which reversed the magnetization of the particles in the sample. According to the exponential decay function, e^{-t/T1}, the magnetization at T₁ is 1/e, or approximately ⅓ less than the equilibrium magnetization. Hence, by extrapolating the time at this magnetization, it was possible to determine the T₁ relaxation rate. This principle also applied to MRI, where 0.2-0.4 ml of nanoemulsion was filled into the one ml syringe and run through a phantom Brucker 500 MHz MRI machine in a 4.7 Tesla magnetic field, giving MRI scans showing nanoemulsion-generated contrast as well as T₁ time measurements.

Cellular Uptake Studies

MCF-7 estrogen-positive human breast adenocarcinoma cells, kindly provided by Dr. Michael Seiden, Massachusetts General Hospital (Boston, Mass.), were maintained in 25 cm³ culture flasks (Corning Inc., Corning, N.Y.) in RPMI®-1640 (Mediatech, Inc., Herndon, Va.) supplemented with 10% heat-inactivated fetal bovine serum (FBS) (Mediatech Inc, Herndon, Va.) and 1% penicillin/streptomycin (Cambrex, Walkersville, Md.), incubated in a humidified air chamber at 37° C. with 5% CO₂. Cells were harvested by brief incubation with 4 ml Trypsin-EDTA (Mediatech Inc., Herndon, Va.) at room-temperature, collected with 8 ml supplemented RPMI®-1640, and centrifuged at 2,000 rpm for 5 minutes. The cell pellet was then re-suspended in 5 ml of supplemented RPMI®-1640 upon which viable cells were visualized by Trypan Blue exclusion and counted using a conventional haemacytometer. Cells were subsequently plated at 5,000 cells per well in 96-well plates (Corning Inc., Corning, N.Y.).

To visualize the uptake of paclitaxel and paclitaxel-loaded nanoemulsion trafficking within the cell, rhodamine-conjugated paclitaxel (Natural Pharmaceuticals, Inc, Beverley, Mass.) was loaded into nanoemulsions. Nanoemulsions were prepared as previously described with the modification that the dye-conjugated drug (6 mg/ml in 1:1 solution of Cremophore EL and pure ethanol) was loaded in the oil phase at 420 μg/ml of the total nanoemulsion volume. MCF cells were allowed to adhere on flame-sterilized glass coverslips within 6-well plates seeded at 5×10⁵ cells/well/2 mL supplemented RPMI. The cells were subsequently incubated with 25 nM solution of rhodamine-paclitaxel solution and rhodamine-paclitaxel encapsulated nanoemulsions for 1, 3 and 6 hour. The treatment was terminated by placing the coverslip, cell side down, onto a glass slide with 50 μL of fluoromount fixing medium (Fluromount-G®) (Southern Biotechnology Associates, Birmingham, Ala.). The slides were allowed to dry for approximately 15 minutes and then stored at 4° C. shielded from light. Rhodamine-fluorescence was then visualized on a fluorescence microscope (Olympus BX61W1) utilizing rhodamine filter. Images were obtained and analyzed with IP Lab v.3.6 software. All treatments were duplicated.

Cytotoxicity of Paclitaxel-Containing Nanoemulsions

Following overnight incubation, allowing the cells to adhere, cells were subjected to treatment with various doses of paclitaxel aqueous solution, plain nanoemulsions, and paclitaxel-loaded nanoenulsions, diluted in supplemented RPMI®-1640. Paclitaxel injectable formulation, Onxol® (Ivax Pharmaceuticals, Miami, Fla.), was used as the source of paclitaxel for delivery as free drug. Paclitaxel stock was then prepared at 10 μM in RPMI®-1640 and subsequently diluted in RPMI®-1640 before use. Stock solutions for paclitaxel were prepared at the same concentrations as their corresponding free-drug counterparts by diluting the nanoemulsions directly in RPMI-1640. Each well received an equivalent of 25 nM paclitaxel concentration (5-times the IC₅₀ value for MCF-7 cells) in the control and test formulations. Treatment with supplemented RPMI®-1640 was used as a negative control and treatment with polyethyleneimine (Mol. wt 10 kDa), a cationic cytotoxic polymer, was used as a positive control. Treatment with the control and test formulations proceeded for 6 days, followed by cell viability was determined by MTS cell viability assay.

MTS cell viability reagent was prepared by dissolving 38 mg of 3-(4,5-dimethylthiazol-2-yl)-5-(3-carboxymethoxyphenyl)-2-(4-sulfophenyl)-2H-tetrazolium (MTS) (Promega, Madison, Wis.) and 2 mg phenazine ethosulfate (PES) (ICN, Aurora, Ohio) in 20 mL Dulbecco's PBS (pH 6.0). After the 6-day treatment period, cell medium was aspirated and replaced with 1:10 diluted MTS reagent in supplemented RPMI-1640. Cells were incubated for at least 4 hours at 37° C., after which plates were read at 490 nm with a Biotek Synergy HT plate reader, where the quantity of formazan product as measured by the amount of 490 nm absorbance is directly proportional to the remaining number of living cells.

From the particle characterization analysis, the standard nanoemulsions had a diameter of 90.4±5.1 nm and a surface charge of −24.9±1.6 mV. In comparison, the stearylamine nanoemulsions had average diameters of 100.2±9.1 nm and a surface charge of +21.7±2.4 mV. Thus, while particle size did not change dramatically when the stearylamine was used as a co-surfactant, the surface charge became positive. This may help to improve cell-nanoemulsion interaction due to the electrostatic attractions of the positively-charged emulsion droplets with the negatively-charged cell surfaces. Freeze fracture electron microscopic imaging of standard nanoemulsions containing stearylamine showed that the size of the nanoemulsion droplets ranged from 25 to 90 nm with some aggregates ranging in size from 100 to 300 nm.

T₁ relaxation time measurements by nuclear magnetic resonance (NMR) allowed for speculation on the contrast agent capabilities of the nanoemulsion formulations. Relating the concentration of Gd³⁺ with the reciprocal of T₁ results in a linear plot having a slope of 9.2 sec⁻¹ mmol⁻¹. This slope is referred to as R₁ and indicates a material's relative contrast efficiency, since a higher slope signifies enhanced contrast and lowered T₁ times. R₁ values may be compared, as was done with the Gd³⁺ and DTPA aqueous solution simulating the commercially-marketed Magnevist® contrast agent, which served as the standard basis for comparison. It is clear from these results that gadolinium loaded nanoemulsions significantly reduce the relaxation time relative to pure water, the unloaded nanoemulsion or even the aqueous solution of DTPA-Gd³⁺ complex. Therefore, these nanoemulsions should act as contrast-enhancing agents. At 0.58 mM Gd³⁺ nanoemulsion, the T₁ value was reduced by around 30 fold and at 12.7 mM Gd³⁺ containing nanoemulsion, the T₁ was reduced from 3.022 seconds for pure water to only 8 milliseconds.

An analogous plot of Gd³⁺ versus 1/T₁ measurements for the Magnevist®-like solution gives a slope of 3.3 sec⁻¹ mmol⁻¹. The higher slope of the nanoemulsions as compared with that of the Gd³⁺-DTPA solution suggests that the nanoemulsions may function as comparable, if not better, contrast agents. Furthermore, the control samples containing only DTPA-PE added to the nanoemulsions did not significantly change the relaxation time. Their T₁ relaxation time was 2.8±0.08 seconds, a statistically insignificant decrease from the 3.02 seconds of pure water. Conversely, the addition of only Gd³⁺ to the nanoemulsions did not act to decrease relaxation times, signifying that Gd³⁺ ions need to be anchored onto the oil droplets with DTPA-PE in order to interact with water protons and decrease relaxation times.

Phantom MRI was also employed to assess T₁ relaxation times and visualize the contrast provided by the nanoemulsions. For these results, the plot of Gd³⁺ concentration versus 1/T₁ measurements gives a slop of 3.4 sec⁻¹ mmol⁻¹. It can be noted that the R₁ for this data is not comparable to the measurements extrapolated from NMR. Because the two machines differed in their magnetic field strengths (11.7 Tesla for NMR, and 4.7 Tesla for MRI), recovery time depended not only on the Gd³⁺ concentration, but also on the magnetic field strength (Fellner et al., 2002).

An MRI scan of the nanoemulsion samples containing different concentrations of gadolinium was carried out with a time-of-repetition of 50 milliseconds after magnetic pulsing. Brightened images of Gd³⁺-loaded nanoemulsions were observed, demonstrating the contrast agent capability of the system. As the concentration of Gd³⁺ increased, the image appeared brighter due to the lowered relaxation rate. However, at 12.7 mM Gd³⁺, the image looked darker than that of the other concentrations, for it is likely that the system was already saturated with gadolinium.

In vitro studies were performed to test the nanoemulsions in a living system and were imaged by cell fluorescent microscopy. The nanoemulsion system appeared to carry the paclitaxel across the cell membrane based on the fluorescent markers that were observed inside the cells after 1, 3 and 6 hours of incubation.

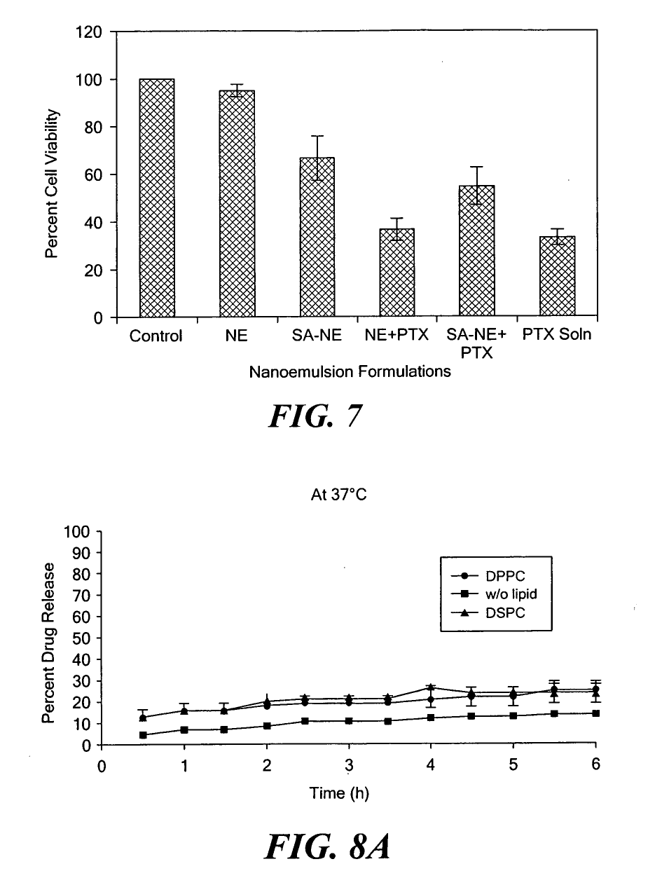

Cell cytotoxicity studies indicated that drug-loaded nanoemulsions had lethal effects on cells, and thus fulfilled both the drug delivery and imaging modalities of a multifunctional system. FIG. 7 shows the percent cell viability for the indivated formulation as measured by light absorbance during the MTS assay. Drug-loaded nanoemulsions (25 nM) or aqueous drug solutions were incubated with MCF-7 carcinoma cells for 6 days. The lower cell viabilities associated with drug-loaded nanoemulsions compared to those of the plain nanoemulsions indicate that paclitaxel delivery to the cells was responsible for the cell death. Thus, the multifunctional capability of these nanoemulsions has been demonstrated in vitro.

### EXAMPLE IV

### Temperature Sensitive Nanoemulsions

Temperature-sensitive nanoemulsions were prepared by the sonication method by incorporating phospholipids that undergo phase transition between 40-45° C. To 4 ml of deionized water, 80 mg of egg phosphatidylcholine (Lipoid® E80, Lipoid® GMBH, Ludwigshfaen, Germany) and 60 mg of either dipalmitoylphosphatidylcholine (DPPC) or distearoylphosphatidylcholine (DSPC) were added. The mixture was stirred for 30 minutes. Separately, a 1 ml aliquot of pine-nut oil was taken. The two phases were then heated separately on a hot plate at 70° C. for 3-5 minutes. The oil phase was added to the aqueous solution and the mixture was sonicated at 21% amplitude and 50% duty cycle (Sonics and Materials Inc., Vibra Cell VC 505, Newtown, Conn.) for 10 minutes resulting in the formation of the nanoemulsion.

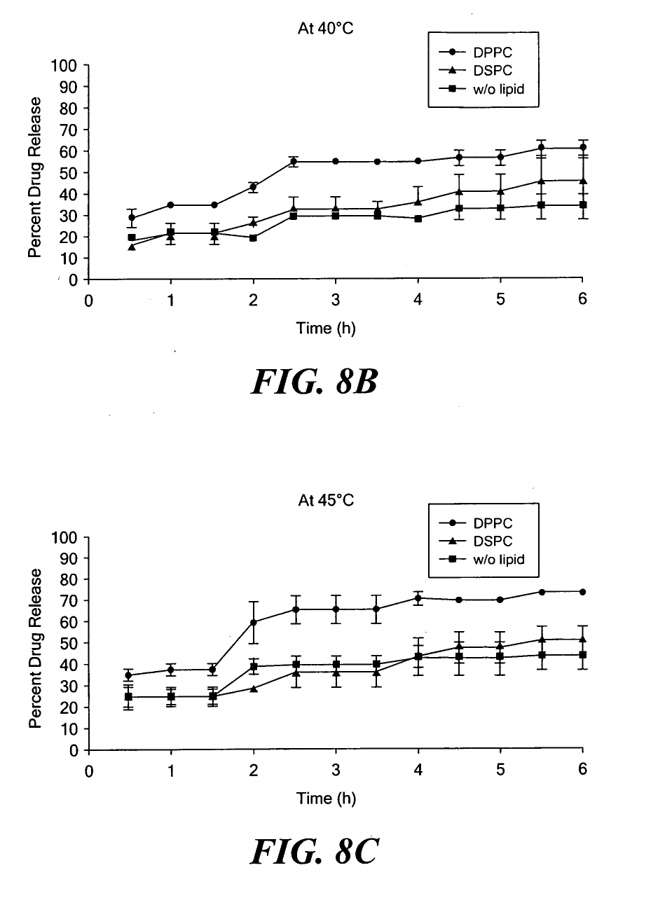

The percent drug release versus time in hours was plotted. As indicated in FIGS. 8A-8C, the nanoemulsion with distearoylphosphatidylcholine (DSPC) as the co-emulsifier showed temperature sensitive drug release, with increasing drug release per the same time period as the temperature was increased from 37° C. to 45° C.

### REFERENCES

Bagwe, R. P., J. R. Kanicky, B. J. Palla, P. K. Patanjali, D. O. Shah, Charman, W. N., J Pharm Sci 89 (2000) 967.
Bardelmeijer, H. A., O. van Tellingen, J. H. Schellens, J. H. Beijnen, Invest New Drugs 18 (2000) 231.
Brannon-Peppas, L., Blanchette, J. O. Nanoparticle and targeted systems for cancer therapy. Adv. Drug Del. Rev., 56, 1649-1659 (2004).
Buszello, K., B. Muller, in F. Nielloud, G. Marti-Mestres (Editors), Pharmaceutical Emulsions and Suspensions, Marcel Dekker, New York, 2000, p. 191.
Crit Rev Ther Drug Carrier Syst 18 (2001) 77.
Fellner, S., Bauer, B., Miller, D. S., Schaffrik, M., Fankhanel, M., Sprub, T., Bernhardt, G., Graeff, C., Farber, L., Gschaidmeier, H., Buschauer, A., Fricker, G. Transport of paclitaxel (Taxol) across the blood-brain barrier in vitro and in vivo. J. Clin. Invest., 110, 1309-1318 (2002).
Gallo, J. M., Li, S., Guo, P., Reed, K., Ma, J. The effect of P-glycoprotein on paclitaxel brain and brain tumor distribution in mice. Cancer Res. 63, 5114-5117 (2003).
Gao, P., B. D. Rush, W. P. Pfund, T. Huang, J. M. Bauer, W. Morozowich, M. S. Kuo, M. J. Hageman, J Pharm Sci 92 (2003) 2386.
Gaysorn, C., R. T. Lyons, M. V. Patel, S. L. Hem, J Pharm Sci 88 (1999) 454.
Gershanik, T., S. Benita, Eur J Pharm Biopharm 50 (2000) 179.
Gimi, B., Pathak, A. P., Ackerstaff, E., Glunde, K., Artemov, D., Bhujwalla, Z. M. Molecular imaging of cancer: applications of MR methods. Proceedings of the IEEE. 93, 784-795 (2005).
Grant, C. W. M., Karlik, S., Florio, E. A liposomal MRI contrast agent: phosphatidylethanolamine-DTPA. Mag. Res. Med., 11, 236-243 (1989).
Gulyaev, A. E., Gelperina, S. E., Skidan, I. N., Antropov, A. S., Kivman, G. Y., Kreuter, J. Significant transport of doxorubicin into the brain with polysorbate 80-coated nanoparticles. Pharm. Res, 16, 1564-1569 (1999).
Gursoy, N., J. S. Garrigue, A. Razafindratsita, G. Lambert, S. Benita, J Pharm Sci 92 (2003) 2411.
Gursoy, R. N., S. Benita, Biomed Pharmacother 58 (2004) 173.
Hansrani, P. K., Davis, S. S., Groves, M. J. The preparation and properties of sterile intravenous emulsions. J. Parenteral Sci. Technol., 37, 145-150 (1983).
Haskell, R., www.banyu-zaidan.or.jp/symp/soyaku/haskell.pdf, date 45 accessed, Oct. 6, 2005.
Khoo, S. M., A. J. Humberstone, J. H. Porter, G. A. Edwards, W. N. Charman, Int. J. Pharm. 167 (1998) 155.
Lanza, G. M., Ramerich, R., Caruthers, S., Wickline, S. A. Molecular imaging in MR with a targeted paramagnetic nanoparticles. Medica Mundi., 63, 5838-5843 (2003).
Lazarou J., Pomeranz, B. H., Corey, P. N. Incidence of adverse drug reactions in hospitalized patients. J. Am. Med. Assoc., 15, 1200-1205 (1998).
Leach, M. O. Breast imaging technology: application of magnetic resonance imaging to angiogenesis in breast cancer. Breast Cancer Res., 3, 22-27 (2001).
Lee, J., S. C. Lee, G. Acharya, C. J. Chang, K. Park, Pharm Res 20 (2003) 1022.
Levchenko, T. S., Rammohan, R., Lukyanov, A. N., Whiteman, K. R., Torchilin, V. P. Liposome clearance in mice: the effect of a separate and combined presence of surface charge and polymer coating. Int. J. Pharm., 240, 95-102 (2002).
Lundberg, B. Preparation of drug-carrier emulsions stabilized with phosphatidylcholine-surfactant mixtures. J. Pharm. Sci., 83, 72-75 (1994).
Malingre, M. M., J. H. Beijnen, H. Rosing, F. J. Koopman, O. van Tellingen, K. Duchin, W. W. Ten Bokkel Huinink, M. Swart, J. Lieverst, J. H. Schellens, Cancer Chemother Pharmacol 47 (2001) 347.
Mathew, A. E., M. R. Mejillano, J. P. Nath, R. H. Himes, V. J. Stella, J Med Chem 35 (1992) 145.
Misra, A., Ganesh, S., Shahiwala, A., Shah, S. P. Drug delivery to the central nervous system: a review. J. Pharm. Pharmaceutical Sci., 6, 252-273 (2003).
Oyewumi, M. O., Yokel, R. A., Jay, M., Coakley, T., Mumper, R. J. Comparison of cell uptake, biodistribution and tumor retention of folate-coated and PEG-coated gadolinium nanoparticles in tumor-bearing mice. J. Control. Rel., 95, 613-626 (2004).
Oyewumi, M. O., Liu, S., Moscow, J. A., Mumper, R. J. Specific association of thiamine-coated gadolinium nanoparticles with human breast cancer cells expressing thiamine transporters. Bioconj. Chem., 14, 404-411 (2003).
Pan, G., Shawer, M., Oie, S., Lu, D. R. In vitro gene transfection in human glioma cells using a novel and less cytotoxic artificial lipoprotein delivery system. Pharm. Res., 20, 738-744 (2003).
Piemi, M. P., D. Korner, S. Benita, MartyJp, J Control Release 58 (1999) 177.
Poelma, F. G., R. Breas, J. J. Tukker, D. J. Crommelin, J Pharm Pharmacol 43 (1991) 317.
Pouton, C. W., Adv. Drug Deliv. Rev. 25 (1997) 47.
Pouton, C. W., Eur J Pharm Sci 11 Suppl 2 (2000) S93.
Pubmed, Paclitaxel, http://www.ncbi.nlm.nih.gov/entrez/query.fcgi?cmd=Retrieve&d b=mesh&list_uids=68017239&dopt=Full (site visited Jul. 31, 2005).
Reynolds, C. H., Annan, N., Beshah, K., Huber, J. H., Shaber, S. H., Lenkinski, R. E., Wortman, J. A. Gadolinium-loaded nanoparticles: new contrast agents for magnetic resonance imaging. J. Am. Chem. Soc., 122, 8940-8945 (2000).
Sakai, M., T. Imai, H. Ohtake, H. Azuma, M. Otagiri, J Pharm Pharmacol 51 (1999) 27.
Salager, J.-L., in F. Nielloud, G. Marti-Mestres (Editors), Pharmaceutical Emulsions and Suspensions, Marcel Dekker, New York, (2000), p. 19.
Schroeder, U., Sommerfeld, P., Sabel, B. A. Efficacy of oral dalargin-loaded nanoparticle delivery across the blood brain barrier. Peptides, 19, 777-780 (1998).
Schroeder, U., Sommerfeld, P., Ulrich, S., Sabel, B. A. Nanoparticle technology for delivery of drugs across the blood brain barrier. J. Pharm. Sci., 87, 1305-1307 (1998).
Silvestri, S., Ganguly, N., Tabibi, E. Predicting the effect of nonionic surfactants on dispersed droplet radii in submicron oil-in-water emulsions. Pharm. Res., 9, 1347-1350 (1992).
Singla, A. K., A. Garg, D. Aggarwal, Int J Pharm 235 (2002) 179. Malingre, M. M, J. H. Beijnen, J. H. Schellens, Invest New Drugs 19 (2001) 155.
Straubinger, R. M., A. Sharma, M. Murray, E. Mayhew, J Natl Cancer Inst Monogr (1993) 69.
Takahashi, M., Uematsu, H., Hatabu, H., MR imaging at high magnetic fields. Eur. J. Radiology, 46, 45-52 (2003).
Torchilin, V. P. PEG-based micelles as carriers of contrast agents for different imaging modalities. Adv. Drug Del. Rev., 54, 235-252 (2002).
Torchilin, V. P. Polymeric contrast agents for medical imaging. Curr. Pharm Biotech., 1, 183-215 (2000).
University of Maryland Medical Center, Gamma-linolenic acid, http://www.umm.edu/altmed/ConsSupplements/GammaLinolenicAcid GLAcs.html (site visited Jul. 17, 2005).
Uno, T., T. Kazui, Y. Suzuki, H. Hashimoto, K. Suzuki, B. A. Muhammad, Lipids 34 (1999) 249.
Wasan, K. M., Drug Dev Ind Pharm 27 (2001) 267.
Weissig, V., Babich, J., Torchilin, V. Long-circulating gadolinium-loaded liposomes: potential use for magnetic resonance imaging of the blood pool. Colloids and Surfaces B: Biointerfaces, 18, 293-299 (2000).
Wickline, S. A., Lanza, G. M. Nanotechnology for molecular imaging and targeted therapy. Circulation, 107, 1092-1095 (2003).
Yang, S. C., S. Benita, Drug Dev Res 50 (2000) 476.
Yang, S., R. N. Gursoy, G. Lambert, S. Benita, Pharm Res 21 (2004) 261.
Zhao, D. L., B. H. Hirst, Digestion 47 (1990) 200.

While the present invention has been described in conjunction with a preferred embodiment, one of ordinary skill, after reading the foregoing specification, will be able to effect various changes, substitutions of equivalents, and other alterations to the compositions and methods set forth herein. It is therefore intended that the protection granted by Letters Patent hereon be limited only by the definitions contained in the appended claims and equivalents thereof.

What is claimed is:

1. An oil-in-water nanoemulsion delivery system for a therapeutic, monitoring or diagnostic agent, said delivery system comprising:
an oil phase comprising at least one oil and having a concentration of greater than or equal to 2% (w/w) of at least one polyunsaturated fatty acid;
an emulsifier phase comprising at least one emulsifier; and
an aqueous phase,
wherein one or more therapeutic, monitoring and/or diagnostic agents are dispersed in said oil phase.

2. The delivery system of claim 1, wherein said at least one oil is selected from the group consisting of flax seed, safflower, pine nut, primrose, black currant, borage, wheat germ, chia, hemp, perilla, grape, squalene and fungal oils.

3. The delivery system of claim 1, wherein said at least one polyunsaturated fatty acid is of the omega-3 or omega-6 family.

4. The delivery system of claim 3, wherein said at least one polyunsaturated fatty acid comprises linolenic acid.

5. The delivery system of claim 3, wherein said at least one polyunsaturated fatty acid comprises linoleic acid.

6. The delivery system of claim 1, wherein said oil phase is in the form of oil droplets having an average size of from 5-500 nm dispersed in said aqueous phase.

7. The delivery system of claim 1, wherein the concentration of said at least one polyunsaturated fatty acid in said oil phase is greater than or equal to 5% (w/w).

8. The delivery system of claim 1, wherein the concentration of said at least one polyunsaturated fatty acid in said oil phase is greater than or equal to 10% (w/w).

9. The delivery system of claim 1, wherein the concentration of said at least one polyunsaturated fatty acid in said oil phase is between 20% and 80% (w/w).

10. The delivery system of claim 1, wherein the concentration of said oil phase in said nanoemulsion is between 5% and 40% (w/w).

11. The delivery system of claim 1, wherein the concentration of said oil phase in said nanoemulsion is between 10% and 30% (w/w).

12. The delivery system of claim 1, wherein said at least one emulsifier is selected from the group consisting of neutral, positively charged, and negatively charged natural or synthetic phospholipid molecules.

13. The delivery system of claim 12, wherein said at least one emulsifier is a natural phospholipid selected from the group consisting of soybean lecithin, egg lecithin, phosphatidylglycerol, phosphatidylinositol, phosphatidylethanolamine, phosphatidic acid, sphingomyelin, diphosphatidylglycerol, phosphatidylserine, phosphatidylcholine and cardiolipin.

14. The delivery system of claim 12, wherein said at least one emulsifier is a synthetic phospholipids selected from the group consisting of dimyristoylphosphatidylcholine, dimyristoylphosphatidylglycerol, distearoylphosphatidylglycerol and dipalmitoylphosphatidylcholine.

15. The delivery system of claim 12, wherein said at least one emulsifier is selected from the group consisting of hydrogenated or partially hydrogenated lecithins and phospholipids.

16. The delivery system of claim 1, wherein the concentration of said emulsifier phase in said nanoemulsion is between 0.5% and 15% (w/v).

17. The delivery system of claim 1, wherein the concentration of said emulsifier phase in said nanoemulsion is between 1% and 10% (w/v).

18. The delivery system of claim 1, wherein said aqueous phase comprises purified or ultrapure water, saline, buffered saline, buffered aqueous phase, glycerine, or low molecular weight polyethylene glycol, or combinations thereof.

19. The delivery system of claim 1, wherein the concentration of said aqueous phase in said nanoemulsion is between 30% and 90% (w/w).

20. The delivery system of claim 1, wherein said nanoemulsion further comprises a pharmaceutical aid selected from the group consisting of stabilizers, preservatives, buffering agents, antioxidants, polymers, proteins and charge inducing agents.

21. The delivery system of claim 1, wherein said one or more therapeutic agents are selected from the group consisting of nonsteroid anti-inflammatory compounds, antineoplastic compounds, antibiotics, anticonvulsants, antiepileptics, antifungals, glycosaminoglycans, hypnotics, vitamins, beta-adrenergic antagonists, antianxiety agents, tranquilizers, antidepressants, corticorsteroids, anabolic steroids, estrogens and progesterones.

22. The delivery system of claim 1, wherein said one or more diagnostic and/or monitoring agents are selected from the group consisting of compounds used for magnetic resonance imaging contrast enhancement, computed tomography contrast enhancement, x-ray contrast enhancement, ultrasound contrast enhancement and optical imaging; and radioactive isotopes for gamma and positron emission tomography imaging.

23. The delivery system of claim 1, wherein said one or more therapeutic, diagnostic and/or monitoring agents are substantially hydrophobic.
